(19) United States
(12) Patent Application Publication
(10) Pub. No.: US 2002/0064523 A1
(43) Pub. Date: May 30, 2002

(54) SYNTHETIC MULTIVULVA (SYNMUV) POLYPEPTIDES

(76) Inventors: H ROBERT HORVITZ, AUBURNDALE, MA (US); CRAIG CEOL, SOMERVILLE, MA (US); XIAOWEI LU, ARLINGTON, MA (US)

Correspondence Address: KRISTINA BIEKER- BRADY, CLARK & ELBING 176 FEDERAL STREET, BOSTON, MA, 02110

(21) Appl. No.: 09/220,091

(22) Filed: Dec. 23, 1998

Related U.S. Application Data
(63) Continuation-in-part of application No. 09/087,136, filed on May 28, 1998.
(60) Provisional application No. 60/047,996, filed on May 28, 1997.

(51) Int. Cl. A61K 38/43

(52) U.S. Cl. 424/94.1

(57) ABSTRACT

The invention provides novel genes involved in cell fate and cell proliferation, including lin-37, lin-35, lin-53, lin-55, lin-52, and lin-54 in multiple species and an E2F-1 gene of C. elegans. Methods for utilizing the genes and encoded proteins are also provided.

### CROSS REFERENCE TO RELATED APPLICATIONS

This application is a continuation-in-part of Ser. No. 09/087,136, filed May 28, 1998, which claims priority from U.S. provisional application serial No. 60/047,996, filed May 28, 1997.

### BACKGROUND OF THE INVENTION

The field of the invention is cell proliferation.

Previously we have identified and studied the synthetic multivulva genes in the nematode Caenorhabditis (C.) elegans. C. elegans is well suited to developmental genetic studies because the entire cell lineage has been mapped and is essentially invariant from one animal to the next. Thus, by comparing the cell lineage of a wild-type animal to that of a mutant animal, the changes in cellular fates caused by the mutation can be determined.

A number of mutations that alter cell lineage, termed lin mutations, were obtained in genetic screens conducted by Horvitz and Sulston in the late 1970's. A subset of the mutations affected the formation of the vulva, a structure on the ventral surface of C. elegans hermaphrodites through which eggs are laid and through which sperm enters during cross-fertilization. Six vulval precursor cells have the potential to undertake a vulval cell lineage as defined by the number and pattern of cell divisions. In a wild type animal only three of these cells actually undertake vulval cell fates and these three cells generate the 22 cells that make up the adult vulva. In multivulva (Muv) animals, most or all of the six vulval precursor cells undertake vulval cell fates. In addition to the cells required for the formation of a normal vulva, these mutant animals generate an excess of cells which cause the formation of raised, vulva-like structures on the ventral surface of the animal. On the other hand, a vulvaless (Vul) phenotype results when no or too few vulval precursor cells adopt vulval cell fates.

Genetic and molecular analyses of Muv and Vul animals have defined a Ras signal transduction pathway that mediates induction of the hermaphrodite vulva. This pathway includes the LIN-3 EGF-like ligand, the LET-23 receptor tyrosine kinase, the SEM-5 adaptor, LET-60 Ras, the KSR-1 kinase, LIN-45 Raf, MEK-2, and the MPK-1 MAP kinase, and regulates the activities of the ETS transcription factor LIN-1 and the winged-helix transcription factor LIN-31 (reviewed by Horvitz and Stemberg, Nature. 351:535-41, 1991; Sundaram and Han, Bioessays 18:473-480, 1996; Tan et al., Cell 93:569-580 1998). Mutant animals in which this pathway is ectopically activated can display a Muv phenotype, whereas mutant animals that have reduced Ras pathway signalling can display a Vul phenotype.

As in the worm, Ras pathways have been found to control cell proliferation in a range of organisms from the yeast S. cerevisiae to humans. The Ras pathway defines one class of oncogene signaling pathways; members of this pathway, most commonly Ras itself, have been shown to be mutated in a broad range of human cancers (Hunter, Cell 88:333-346, 1997).

The synthetic multivulva (synMuv) genes act in two functionally-redundant pathways as negative regulators of worm signalling pathway. The first synthetic multivulva mutant was identified by Horvitz and Sulston. The two genetic loci mutated in this mutant were termed lin-8 and lin-9. Reduction-of-function mutations in both of these loci were required for a multivulva phenotype. Subsequent genetic screens identified a set of loci which fall into the same class as lin-8, termed class A genes, and genes which fall into the same class as lin-9, termed class B genes. In general, an animal with a reduction-of-function mutation in any class A gene and a reduction-of-function mutation in any class B gene will display a multivulva phenotype, while animals carrying one or mutations of the same class have a wild-type vulval phenotype. These two mutations appear to define two functionally redundant pathways that negatively regulate the expression of vulval cell fates.

Thus far four class A loci (lin-8, lin-15A, lin-38 and lin-56) and ten class B loci (lin-9, lin-15B, lin-35, lin-36, lin-37, lin-51, lin-52, lin-53, lin-54 and lin-55) have been identified genetically. lin-15 encodes both A and B activities in two non-overlapping transcripts. lin-15A lin-15B, lin-36, and lin-9 have been cloned and encode novel proteins (Clark et al., Genetics 137:987-997, 1994; Huang et al., Mol. Biol. Cell 5:395-411, 1994; Beitel, Genetic and molecular analyses of let-60 ras, lin-1 and lin-9: genes that function in C. elegans vulval induction. Ph.D. Thesis, Massachusetts Institute of Technology, Cambridge, Mass., 1994).

### SUMMARY OF THE INVENTION

We have cloned synMuv genes in C. elegans that are part of a pathway which may be used as a genetic and biochemical model system for tumor suppression and, conversely, cancer. Specifically, we have now cloned lin-35, lin37, lin-53, lin-52, lin-54 and lin-55. Upon sequencing of lin-35, lin-53, and lin-55, we discovered that all three genes have homology to proteins in the retinoblastoma tumor suppressor pathway family of proteins. This indicates that the synMuv pathway genes and proteins may be used to identify additional genes and proteins which are part of the synMuv pathway (e.g., by finding proteins which interact with known synMuv proteins), to identify genes which are part of the mammalian pathway (e.g., by finding homologs of pathway genes, such as lin-9, lin-15B, lin37, lin-52, or lin-54, which to date have no known mammalian homolog) and to identify therapeutic compounds which modulate this pathway.

The invention also features novel synMuv nucleic acids, proteins, and antibodies which bind these proteins.

In general, the invention features substantially pure nucleic acid (for example, genomic DNA, cDNA, or synthetic DNA) encoding a mammalian synMuv polypeptide, as defined below. In related aspects, the invention also features a vector, a cell (e.g., a nematode, mammalian, yeast, or bacterial cell), and a transgenic animal or embryo thereof which includes such a substantially pure nucleic acid encoding a synMuv polypeptide. C. elegans lin-9 and lin-15B are specifically excluded from the nucleic acid of the invention.

In preferred embodiments, lin-37, lin35, lin-53, lin-55, lin-52, lin-54, or the C. elegans E2F-1 is the gene encoded by the nucleic acid. In most preferred embodiments the gene is a C. elegans gene, a human gene, or murine gene. In other various preferred embodiments, the nucleic acid includes a sequence that hybridizes under high stringency conditions to a probe that is complementary to a synMuv gene such as lin-35, lin-37, lin-52, lin-53, lin54, or lin-55. In preferred embodiments, the probe is 25, 50, 75, 150, or 300 nucleotides.

In other aspects, the invention features a DNA sequence substantially identical to a DNA sequence shown in any one of FIGS. 3, 5, 7, 8, 10, 12, 14, 26, or 27.

In another aspect, the invention features a substantially pure polypeptide having a sequence comprising one of the synMuv amino acid sequences shown in FIGS. 2, 4, 6, 8, 9, 11, or 13 or encoded by a nucleic acid of FIGS. 26 or 27.

In yet another aspect, the invention features a substantially pure DNA which includes a promoter capable of expressing the synMuv gene in a cell. In preferred embodiments, the synMuv gene is lin-37, lin-35, lin-53, lin-55, lin-52, lin-54, or C. elegans E2F-1. C. elegans lin-9 and lin-15B are specifically excluded from the DNA of the invention.

In preferred embodiments, the promoter is the promoter native to a synMuv gene. Additionally, transcriptional and translational regulatory regions are preferably native to a synMuv gene.

In another aspect, the invention features a method of modulating cell death which involves producing a transgenic cell having a transgene encoding a synMuv polypeptide, wherein the transgene is expressed. Preferably, the transgene is integrated into the genome of the cell and is expressed in the cell at a level sufficient to modulate cell death. Preferably, the modulation of cell death is in a mammal and the transgene is a mammalian synMuv gene.

In another aspect, the invention features a method of detecting a synMuv gene in a cell involving: (a) contacting the synMuv gene or a portion thereof greater than 9 nucleic acids, preferably greater than 18 nucleic acids in length with a preparation of genomic DNA from the cell under hybridization conditions providing detection of DNA sequences having about 50% or greater nucleotide sequence identity to the amino acid encoding DNA sequences of lin-37, lin-35, lin53, lin-55, lin-52, lin-54, or C. elegans E2F-1.

In another aspect, the invention features a method of producing a synMuv polypeptide which involves: (a) providing a cell transformed with nucleic acid encoding a synMuv polypeptide positioned for expression in the cell; (b) culturing the cell under conditions for expressing the nucleic acid; and (c) isolating the synMuv polypeptide. In preferred embodiments the expression of the nucleic acid encoding the synMuv polypeptide is regulated by a constitutive or inducible promotor. In one embodiment, the promotor is a heterologous promotor.

In another aspect, the invention features substantially pure synMuv polypeptide. In a preferred embodiment, the polypeptide includes a greater than 50 amino acid sequence substantially identical to a greater than 50 amino acid sequence shown in any one of FIGS. 2, 4, 6, 8, 9, 11, or 13 or encoded by a nucleic acid of FIG. 26 or 27. In another preferred embodiment, the polypeptide includes a polypeptide encoded by DNA which hybridizes at high stringency to at least a portion of a sequence chosen from SEQ ID NOS: 17, 18, and 19. Most preferably, the polypeptide has at least one biological activity of a synMuv protein of the foregoing figures.

In another aspect, the invention features a synMuv gene isolated according to the method involving: (a) providing a cell sample; (b) introducing by transformation into the cell sample a candidate synMuv gene; (c) expressing the candidate synMuv gene within the cell sample; and (d) determining whether the cell sample exhibits an altered cell proliferative, whereby a response identifies a synMuv gene.

In another aspect, the invention features a method of identifying a synMuv gene in a cell, involving: (a) providing a preparation of cellular DNA (for example, from the human genome or a cDNA library (such as a cDNA library isolated from a cell type that is cancerous); (b) providing a detectably-labelled DNA sequence (for example, prepared by the methods of the invention) having homology to a conserved region of a synMuv gene or to a conserved region of lin-9 or lin-15B; (c) contacting the preparation of cellular DNA with the detectably-labelled DNA sequence under hybridization conditions providing detection of genes having 50% nucleotide or greater sequence identity; and (d) identifying a synMuv gene by its association with the detectable label.

In another aspect, the invention features a method of identifying a synMuv gene involving: (a) providing a cell tissue sample; (b) introducing by transformation into the cell sample a candidate synMuv gene; (c) expressing the candidate synMuv gene within the cell sample; and (d) determining whether the cell sample exhibits alteration in cell proliferation, whereby a change in (i.e. modulation of) cell proliferation identifies a synMuv gene.

Preferably, the cell sample is a cell type which may be assayed for proliferation or a synMuv phenotype; the candidate synMuv gene is obtained from a cDNA expression library; and the phenotype involves cell proliferation.

In another aspect, the invention features a method of modulating cell proliferation in an animal wherein the method includes: (a) providing DNA encoding at least one synMuv polypeptide to a cell; wherein the DNA is integrated into the genome of the cell and is positioned for expression in the cell; and the synMuv gene is under the control of regulatory sequences suitable for controlled expression of the gene(s); wherein the synMuv transgene is expressed at a level sufficient to affect cell proliferation relative to a cell lacking the synMuv transgene. It will be appreciated that synMuv polypeptides also may be administered directly to modulating cell proliferation.

In a related aspect, the invention features a method of modulating cell proliferation wherein the method involves: (a) producing a cell having integrated in the genome a transgene containing the synMuv gene under the control of a promoter providing constitutive expression of the synMuv gene.

In yet another related aspect, the invention features a method of modulating cell death wherein the method involves: (a) producing a cell having integrated in the genome a transgene containing the synMuv gene under the control of a promoter providing controllable expression of the synMuv gene; and (b) regulating the environment of the cell so that the synMuv transgene is controllably expressed in the cell. In preferred embodiments, the synMuv gene is expressed using a tissue-specific or cell type-specific promoter, or by a promoter that is activated by the introduction of an external signal or agent, such as a chemical signal or agent.

In a related aspect, the invention provides a method of modulating cell proliferation in an animal by providing an cell proliferation modulation amount of synMuv polypeptide.

In another aspect, the invention features a purified antibody which binds specifically to a synMuv family protein. Such an antibody may be used in any standard immunodetection method for the identification of a synMuv polypeptide. Preferably, the antibody binds specifically to LIN-37, LIN-35, LIN-53, LIN-55, LIN-52, LIN-54, or C. elegans E2F-1. In various embodiments the antibody may react with other synMuv polypeptides or may be specific for one or a few synMuv polypeptides. The antibody may be a monoclonal or polyclonal antibody.

In another aspect, the invention features a method of identifying a compound which modulates cell proliferation. The method generally includes the steps of (a) providing a cell expressing a gene operably linked to a synMuv gene promoter; (b) contracting the cell with a candidate compound, and (c) monitoring the expression of the gene. An alteration in the level of expression of the synMuv gene indicates the presence of a compound which modulates cell proliferation. The compound may be an inhibitor or an enhancer of cell proliferation.

In preferred embodiments, the gene used in the method is a synMuv gene. In other preferred embodiments, the gene used in the method is a reporter gene. Exemplary reporter genes include, without limitation, gfp, lacZ, luciferase, or CAT.

In preferred embodiments, the gene promoter utilized in the methods of the invention is derived from the lin-35, lin-37, lin-52, lin-53, lin-54, lin-55, or C. elegans E2F-1 gene, or from the lin-9 or lin-15B gene.

Typically, the expression of the gene is measured by assaying the RNA or protein levels (or both) of the expressed gene. For example, the polypeptide expressed by the synMuv gene or by the reporter gene produces a detectable signal under suitable conditions. Quantitatively determining the amount of signal produced requires comparing the amount of signal produced to the amount of signal produced in the absence of any compound being tested or upon contacing the cell with any other compound. The comparison permits the identification of the compound as one which causes a change in the detectable signal produced by the expressed gene (e.g., at the RNA or protein level) and thus identifies a compound that is capable of modulating synMuv activity.

In another aspect, the invention features a method for identifying a gene which modulates cell proliferation. The method generally includes the steps of (a) expressing in a cell (i) a first gene operably linked to a synMuv gene promoter and (ii) a second candidate gene or a fragment thereof and (b) monitoring the expression of the first gene, wherein an increase in the expression of the first gene identifies the second candidate gene as a gene which modulates cell proliferation.

Preferably, the first gene includes a synMuv gene (e.g., lin-35 or lin-53). In yet other preferred embodiments, the first gene includes a reporter gene (e.g., lacZ, CAT, or gfp).

In preferred embodiments, the synMuv gene promoter is derived from the lin-35, lin-37, lin-52, lin-54, lin-53, lin-9, lin-15B, or lin-55 gene promoter. Preferably, the expression of the gene utilized in the method of the invention is measured by assaying the protein level of the expressed first gene or by assaying the RNA level of the expressed first gene.

In another aspect, the invention features a therapeutic composition including as an active ingredient a synMuv polypeptide formulated in a physiologically acceptable carrier.

In a preferred embodiment, the synMuv polypeptide of the therapeutic composition is encoded by nucleic acid which hybridizes at high stringency to at least a portion of a sequence chosen from SEQ ID NOS:17, 18, and 19, the portion being at least 25 nucleotides and the polypeptide having at least one synMuv biological activity.

In another aspect, the invention features a method of modulating cell proliferation of a cell, including administering to the cell a proliferation modulating amount of synMuv polypeptide. In a preferred embodiment, the synMuv polypeptide is encoded by nucleic acid which hybridizes at high stringency to at least a portion of a sequence chosen from SEQ ID NOS:17, 18, and 19, the portion being at least 25 nucleotides and the polypeptide having at least one synMuv biological activity.

In another aspect, the invention features a method of identifying a gene which modulates cell proliferation, including (a) expressing in a cell (i) at least a portion of a first gene selected from lin-9 and lin-15B; and (ii) a second candidate gene or a fragment thereof; and (b) monitoring the expression of the first gene, wherein an increase in expression identifies the candidate gene as a gene which modulates cell proliferation. Preferably, the expression of the gene utilized in the method of the invention is measured by assaying the protein level of the expressed first gene or by assaying the RNA level of the expressed first gene.

In a related aspect, the invention features a method of identifying a gene which modulates cell proliferation, including (a) expressing in a cell (i) at least a portion of a first gene, wherein the first gene includes a sequence chosen from SEQ ID NOS: 17, 18, and 19; and (ii) a second candidate gene or a fragment thereof; and (b) monitoring the expression of the first gene, wherein an increase in expression identifies the candidate gene as a gene which modulates cell proliferation. Preferably, the expression of the gene utilized in the method of the invention is measured by assaying the protein level of the expressed first gene or by assaying the RNA level of the expressed first gene.

In another aspect, the invention features a method of identifying a gene which modulates cell proliferation, including (a) expressing in a cell (i) at least a portion of a first gene operably linked to a promoter selected from the lin-9 promoter and the lin-15B promoter; and (ii) a second candidate gene or a fragment thereof; and (b) monitoring the expression of the first gene, wherein an increase in expression identifies the candidate gene as a gene which modulates cell proliferation. In a preferred embodiment, the first gene is a reporter gene. Preferably, the expression of the gene utilized in the method of the invention is measured by assaying the protein level of the expressed first gene or by assaying the RNA level of the expressed first gene.

In another aspect, the invention features a method of diagnosing an animal for the presence of a cell proliferation disease or an increased likelihood of developing a cell proliferation disease, including measuring gene expression in a sample from said animal, wherein the gene hybridizes at high stringency to at least a portion of a sequence chosen from SEQ ID NOS: 17, 18, and 19, the portion being at least 50 nucleotides, and the polypeptide having at least one synMuv biological activity, an alteration in expression relative to a sample from an unaffected animal being an indication that the animal has a cell proliferation disease or increased likelihood of developing a cell proliferation disease. Preferably, the expression of the gene utilized in the method of the invention is measured by assaying the protein level of the expressed first gene or by assaying the RNA level of the expressed first gene.

In another aspect, the invention features a method of diagnosing an animal for the presence of an cell proliferation disease or an increased likelihood of developing a cell proliferation disease, including isolating a sample of nucleic acid from the animal and determining whether said nucleic acid includes a mutated gene, wherein the gene includes a sequence chosen from SEQ ID NOS: 17, 18, and 19, a mutation in the nucleic acid being an indication that the animal has an cell proliferation disease or an increased likelihood of developing a cell proliferation disease.

In another aspect, the invention features a method of identifying a compound which modulates cell proliferation, including providing a cell expressing a gene operably linked to a gene promoter selected from the list of the lin-9 promoter and the lin-15B promoter, contacting the cell with a candidate compound, and monitoring the expression of the gene, an alteration in the level of expression of the gene indicating the presence of a compound which modulates cell proliferation. In a preferred embodiments, the gene is lin-9 or lin-15 or is a reporter gene. Preferably, the expression of the gene utilized in the method of the invention is measured by assaying the protein level of the expressed first gene or by assaying the RNA level of the expressed first gene.

By “synMuv gene” is meant a gene or fragment thereof having about 50% or greater nucleotide sequence identity to at least one of the synMuv amino acid encoding sequences of FIGS. 2, 4, 6, 8, 9, 11, or 13 or encoded by the sequence of FIG. 26 or FIG. 27, or portions thereof. By a “synMuv gene” is also meant any member of the family of genes characterized by their ability to modulate cell proliferation and having at least 10%, preferably 30%, and most preferably 50% amino acid sequence identity to at least one of the synMuv protein described herein. Representative members of the synMuv gene family include the lin-9, lin15B, lin-37, lin-35, lin-53, lin-55, lin-52, lin-54, and E2F-1 gene of C. elegans, and the lin-54 genes of the mouse and human. Other synMuv genes include those comprising at least a portion of sequences of SEQ ID NOS: 17, 18, or 19. SynMuv genes include those which encode polypeptides encoded by ESTs zp44h06.s1, zr79e11.r1, and EST180962. Other synMuv genes include those that encode other substantially pure naturally-occurring polypeptides as well as allelic variants; natural mutants; induced mutants; preferably, such synMuv genes hybridize to any one of the nucleic acid sequences of the invention under high stringency conditions. Less preferably, synMuv genes hybridize to any one of the nucleic acid sequences of the invention under low stringency conditions (e.g., washing at 2× SSC at 40° C. with a probe length of at least 40 nucleotides). C. elegans lin-15B and lin9 are included in the methods of the invention, but are excluded from the compounds of the invention.

lin-54 genes of the invention, in particular, may alternatively be identified as encoding a protein having at least 40% identity to the boxed region in FIG. 13 and encoding at least one of the cysteine motifs shown in Section VIII, below.

Specifically excluded from the synMuv genes of the invention are known retinoblastoma tumor suppressor pathway genes, including the Rb gene, in all species; p107, in human and mouse; and p130 in humans. Also excluded are known E2F genes, including human, murine, and Drosophila E2F genes (e.g., E2F-1, E2F-2, E2F-3, E2F-4, E2F-5, E2F-6).

By “synMuv polypeptide” or “synMuv protein” is meant a polypeptide encoded by a synMuv gene.

By “biological activity” is meant an activity of the synMuv polypetide when expressed or overexpressed, either alone or in combination with another polypeptide, in a cell, which activity is absent in the absence of said synMuv polypeptide. synMuv polypeptides include the LIN-9, LIN-15B, LIN-35, LIN-37, LIN-52, LIN-53, LIN-54, LIN-55, and E2F-1 polypeptides of C. elegans and the LIN-54 polypeptides of the mouse and human. synMuv biological activity includes modulating or altering cell proliferation. Another activity is rescuing (i.e., suppressing) a synMuv mutant phenotype. Less preferably, a synMuv biological activity is binding to other known synMuv polypeptides, in vivo or in vitro. Another synMuv biological activity is binding to an antibody which recognizes a synMuv polypeptide. Finally, a synMuv biological activity is hybridizing to a detectably-labeled probe from a synMuv gene selected from lin-9, lin 15B, lin 35, lin-37, lin-52, lin-53, lin-54, lin-55, and C. elegans E2F-1.

By “modulating cell proliferation” or “altering cell proliferation” is meant increasing or decreasing the number of cells which undergo cell division in a given cell population or altering the fate of a given cell. It will be appreciated that the degree of modulation provided by a synMuv or modulating compound in a given assay will vary, but that one skilled in the art can determine the statistically significant change in the level of cell proliferation which identifies a synMuv or a compound which modulates a synMuv.

By “inhibiting cell proliferation” is meant any decrease in the number of cells which undergo division relative to an untreated control. Preferably, the decrease is at least 25%, more preferably the decrease is 50%, and most preferably the decrease is at least one-fold.

“High stringency” conditions may include hybridization at about 42° C. and about 50% formamide, 0.1 mg/mL sheared salmon sperm DNA, 1% SDS, 2× SSC, 10% Dextran sulfate, a first wash at about 65° C., about 2× SSC, and 1% SDS, followed by a second wash at about 65° C. and about 0.1× SSC. Alternatively, high stringency conditions may include hybridization at about 42° C. and about 50% formamide, 0.1 mg/mL sheared salmon sperm DNA, 0.5% SDS, 5× SSPE, 1× Denhardt's, followed by two washes at room temperature and 2× SSC, 0.1% SDS, and two washes at between 55-60° C. and 0.2× SSC, 0.1% SDS.

By “polypeptide” is meant any chain of amino acids, regardless of length or post-translational modification (e.g., glycosylation or phosphorylation).

By “substantially identical” is meant a polypeptide or nucleic acid exhibiting at least 50%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity may be measured using sequence analysis software on the default setting (i.e., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By a “substantially pure polypeptide” is meant a synMuv polypeptide which has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, synMuv polypeptide. A substantially pure synMuv polypeptide may be obtained, for example, by extraction from a natural source (e.g., a fibroblast, neuronal cell, or lymphocyte cell); by expression of a recombinant nucleic acid encoding a synMuv polypeptide; or by chemically synthesizing the protein. Purity can be measured by any appropriate method, e.g., those described in column chromatography, polyacrylamide gel electrophoresis, or by HPLC analysis.

A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms but synthesized in E. Coli or other prokaryotes.

By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

By “transformed cell” is meant a cell into which (or into an ancestor of which) has been introduced, by means of recombinant DNA techniques, a DNA molecule encoding (as used herein) a synMuv polypeptide.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism. Self replicating units, such as artificial chromosomes, are included.

By “transgenic” is meant any cell which includes a DNA sequence which is inserted by artifice into a cell and becomes part of the genome of the organism which develops from that cell. As used herein, the transgenic organisms are generally transgenic nematodes or mammals (e.g., C. elegans rodents such as rats or mice) and the DNA (transgene) is inserted by artifice into the nuclear genome.

By “transformation” is meant any method for introducing foreign molecules into a cell. Microinjection, lipofection, calcium phosphate precipitation, retroviral deliver, electroporation and biolistic transformation are just a few of the teachings which may be used. For example, Biolistic transformation is a method for introducing foreign molecules into a cell using velocity driven microprojectiles such as tungsten or gold particles. Such velocity-driven methods originate from pressure bursts which include, but are not limited to, helium-driven, air-driven, and gunpowder-driven techniques. Biolistic transformation may be applied to the transformation or transfection of a wide variety of cell types and intact tissues including, without limitation, intracellular organelles, bacteria, yeast, fungi, algae, animal tissue, and cultured cells.

By “positioned for expression” is meant that the DNA molecule is positioned adjacent to a DNA sequence which directs transcription and translation of the sequence (i.e., facilitates the production of, e.g., a synMuv polypeptide, a recombinant protein or a RNA molecule).

By “reporter gene” is meant a gene whose expression may be assayed; such genes include, without limitation, those encoding-glucuronidase (GUS), luciferase, chloramphenicol transacetylase (CAT), green fluorescent protein (GFP), and β-galactosidase.

By “promoter” is meant minimal sequence sufficient to direct transcription. Also included in the invention are those promoter elements which are sufficient to render promoter-dependent gene expression controllable for cell-type specific, tissue-specific or inducible by external signals or agents; such elements may be located in the 5′ or 3′ regions of the native gene.

By “operably linked” is meant that a gene and a regulatory sequence(s) are connected in such a way as to permit gene expression when the appropriate molecules (e.g., transcriptional activator proteins) are bound to the regulatory sequence(s).

By “conserved region” is meant any stretch of six or more contiguous amino acids exhibiting at least 30%, preferably 50%, and most preferably 70% amino acid sequence identity between two or more homologs of a synMuv family member, (e.g., human LIN-54, and nematode LIN-54).

By “detectably-labelled” is meant any means for marking and identifying the presence of a molecule, e.g., an oligonucleotide probe or primer, a gene or fragment thereof, or a cDNA molecule. Methods for detectably-labelling a molecule are well known in the art and include, without limitation, radioactive labelling (e.g., with an isotope such as ³²P or ³⁵S) and nonradioactive labelling (e.g., chemiluminescent labelling, e.g., fluorescein labelling).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody, e.g., a synMuv specific antibody. A purified antibody may be obtained, for example, by affinity chromatography using recombinantly-produced protein or conserved motif peptides and standard techniques.

By “specifically binds” is meant an antibody which recognizes and binds a protein but which does not substantially recognize and bind other molecules in a sample, e.g., a biological sample, which naturally includes protein.

Other features and advantages of the invention will be apparent from the following description of the preferred embodiments thereof, and from the claims.

### BRIEF DESCRIPTION OF THE DRAWINGS

FIG. 1 shows a schematic of a model for synthetic multivulva gene action.

FIG. 2 shows the LIN-37 protein sequence (SEQ ID NO:1).

FIG. 3 shows the lin-37 cDNA sequence (SEQ ID NO:2).

FIG. 4 shows the LIN-35 protein sequence (SEQ ID NO:3).

FIG. 5 shows the lin-35 cDNA sequence (SEQ ID NO:4).

FIG. 6 shows the LIN-53 protein sequence (SEQ ID NO:5).

FIG. 7 shows the lin-53 cDNA sequence (SEQ ID NO:6).

FIG. 8 shows the partial LIN-55 protein sequence (SEQ ID NO:7) and the lin-55 cDNA sequence (SEQ ID NO:8).

FIG. 9 shows the C. elegans E2F-1 protein sequence (SEQ ID NO:9).

FIG. 10 shows the C. elegans E2F-1 cDNA sequence (SEQ ID NO:10).

FIG. 11 shows the LIN-52 protein sequence (SEQ ID No:11).

FIG. 12 shows the lin-52 cDNA sequence (SEQ ID No:12).

FIG. 13 shows the LIN-54 protein sequence (SEQ ID NO:13).

FIG. 14 shows the lin-54 cDNA sequence (SEQ ID NO:14).

FIG. 15 shows a map of the position of lin-52 on LGIII.

FIG. 16 shows a map of the position of lin-55 on LGII.

FIG. 17 shows a picture of an RNA blot demonstrating that lin-37 message is present in both embryonic and mixed stage RNAs.

FIG. 18 shows a diagram of the lin-37 gene structure and the positions of n758 and n2234.

FIG. 19 shows a diagram of lin-37:: GFP fusion.

FIG. 20 shows a diagram of constructs used for lin-37 rescues.

FIG. 21 shows a chart of LIN-37 hydrophobicity.

FIG. 22 shows a diagram of a model for lin-37 function.

FIG. 23 shows a diagram of data obtained using LIN-36, LIN-9, LIN15A, LIN-15B and SNF1 (as a control) in a protein-protein binding assay.

FIG. 24 shows a picture of the strategy for characterization of molecular interaction of the synMuv gene products.

FIG. 25 shows a diagram of the strategy for detection of additional synMuv alleles.

FIG. 26 shows the sequence of the M. musculus cDNA homolog of lin54 (SEQ ID NO:15).

FIG. 27 shows the sequence of the H. sapiens cDNA homolog of lin-54 (SEQ ID NO:16).

FIG. 28 shows the sequence of the H. sapiens EST zr79e11.r1 (SEQ ID NO:17).

FIG. 29 shows the sequence of the H. sapiens EST EST180962 (SEQ ID NO:18).

FIG. 30 shows the sequence of the H. sapiens EST zp44h06.sl (SEQ ID NO:19).

### DETAILED DESCRIPTION OF THE INVENTION

I. Introduction

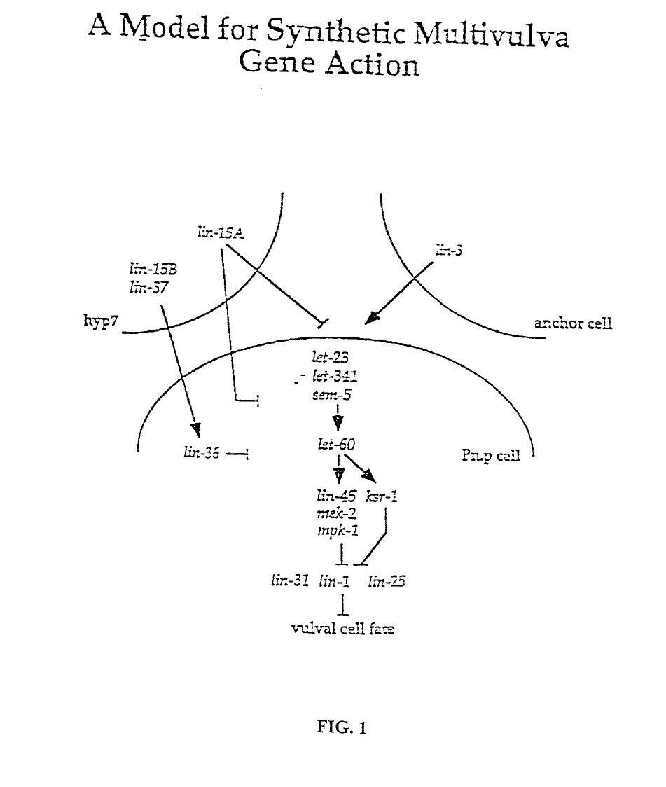

We have cloned lin-37, lin-35, lin-52, lin-53, lin-54, and lin-55. Data obtained from cloning indicates that these class B synMuv genes constitute a tumor suppressor pathway, which is related to a tumor suppressor pathway of clinical importance in humans. Accordingly, the synMuv pathway genes described herein as well as the previously known synMuv genes lin-9 and lin-15B, which had no known mammalian homologs, may be used to identify new tumor suppressors in other species, such as mammals, and may be used to identify therapeutic compounds. The encoded proteins, and the worms may similarly be employed FIG. 1 illustrates the placement of our novel genes within the synMuv pathway.

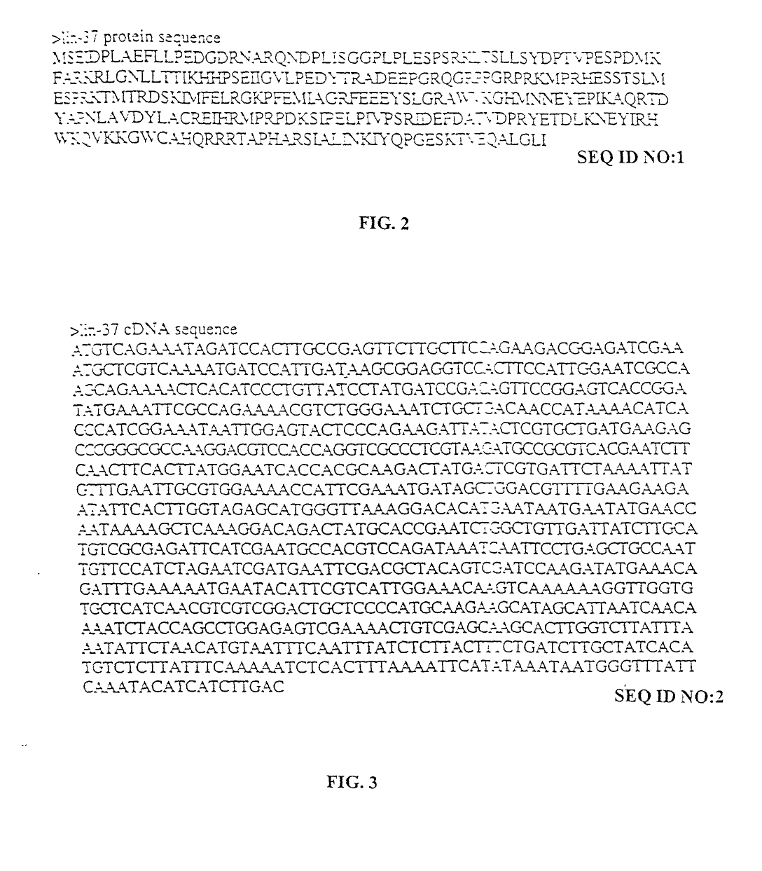

lin-37 encodes a novel protein and, as with the other synMuv genes cloned and described herein, the invention provides the gene (FIG. 2; SEQ ID NO: 1) the protein (FIG. 3; SEQ ID NO: 2), and mutants derived therefrom.

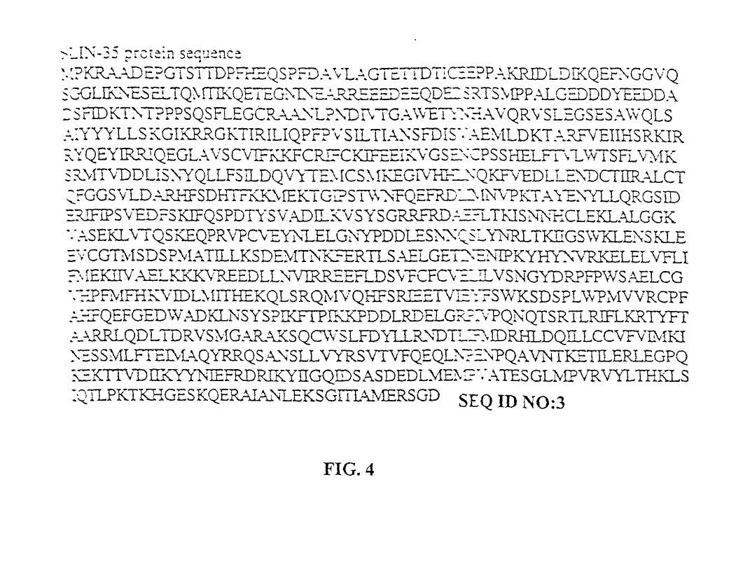

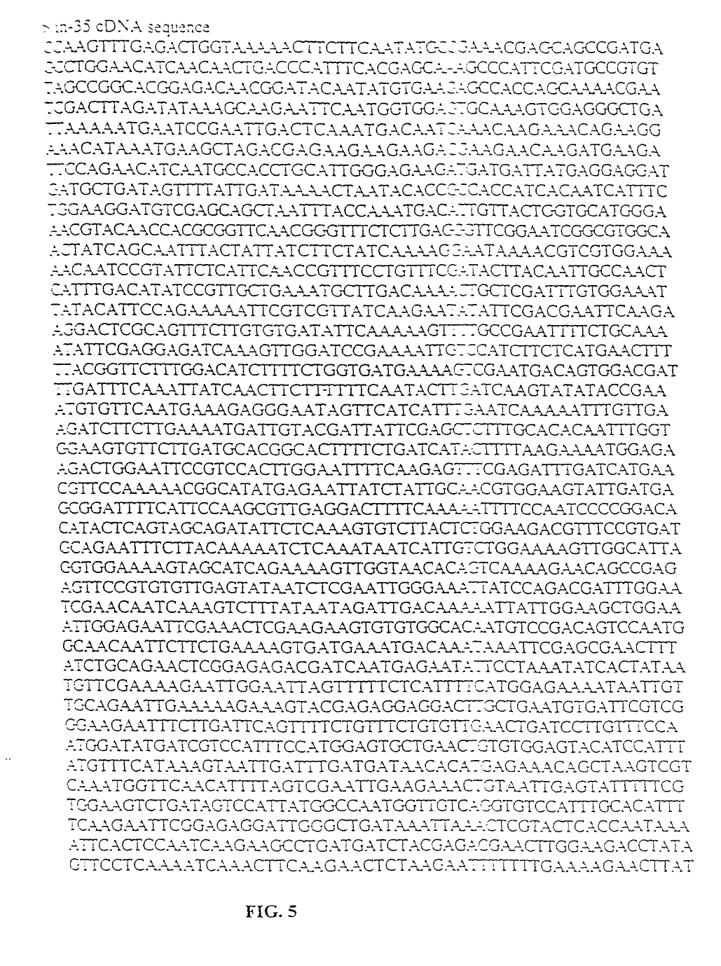

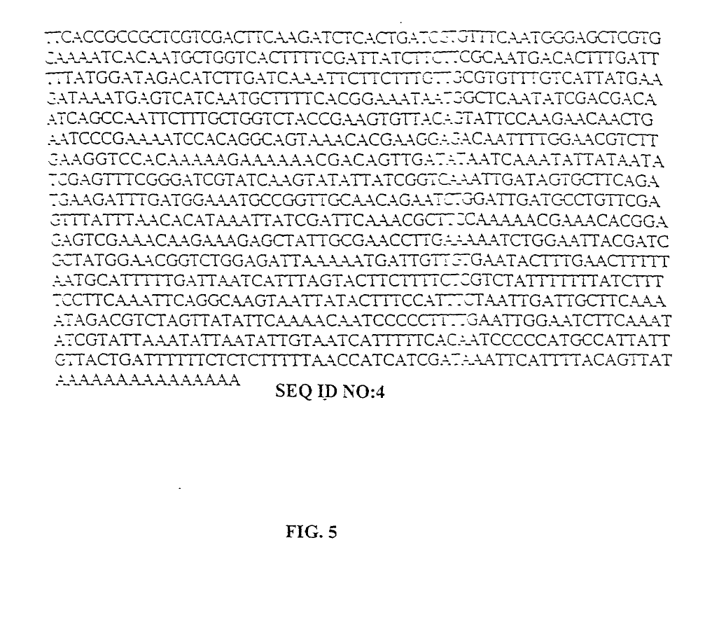

lin-35 has also been cloned. FIG. 4 provides the LIN-35 protein sequence (SEQ ID NO: 3) and FIG. 5 provides the lin-35 sequence (SEQ ID NO: 4). The cloning of lin-35 has shed light on how the class B synMuv genes act to negatively regulate vulval induction. lin-35 encodes a homolog of the mammalian pocket protein family, which includes retinoblastoma protein (Rb), p107, and p130. This family of proteins has been the subject of intense study since the cloning of Rb in 1986. Rb is a tumor suppressor gene; mutations that inactivate Rb predispose individuals to tumor formation. Most commonly, inactivation of Rb results in a type of eye cancer, retinoblastoma, although inactivating mutations in Rb have been found in other types of tumors. The Rb protein is thought to function as a negative regulator of cell cycle progression. A number of molecules which interact, both directly and indirectly, with Rb and the other pocket proteins have been characterized in mammalian cells.

Our cloning of lin-53 (FIGS. 5 and 7) indicates that lin-53 encodes a homolog of p48, a protein which has been shown to bind Rb. Although the functional significance of the interaction between p48 and Rb is not fully understood, recent studies suggest p48 may play a role in remodeling chromatin structure.

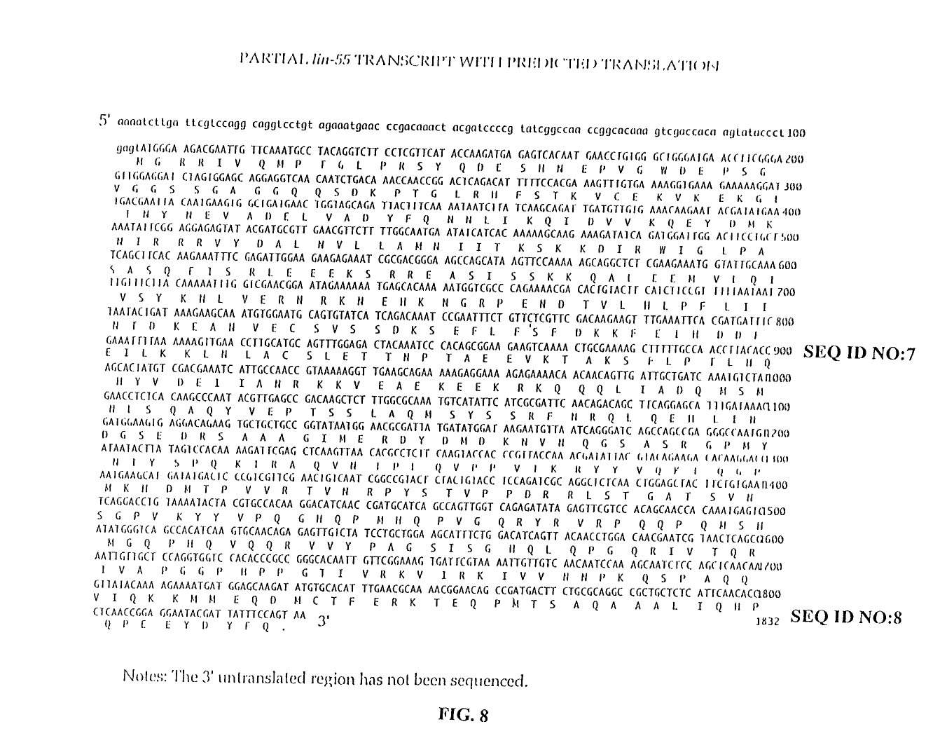

We have also cloned lin-55 (FIG. 8) and found that it encodes a homolog of the DP family of proteins. DP family members, together with E2F proteins, bind DNA at specific sites, thereby regulating the transcription of genes that are essential for cell cycle progression. Pocket proteins such as Rb bind to the DPE2F complex to repress transcription.

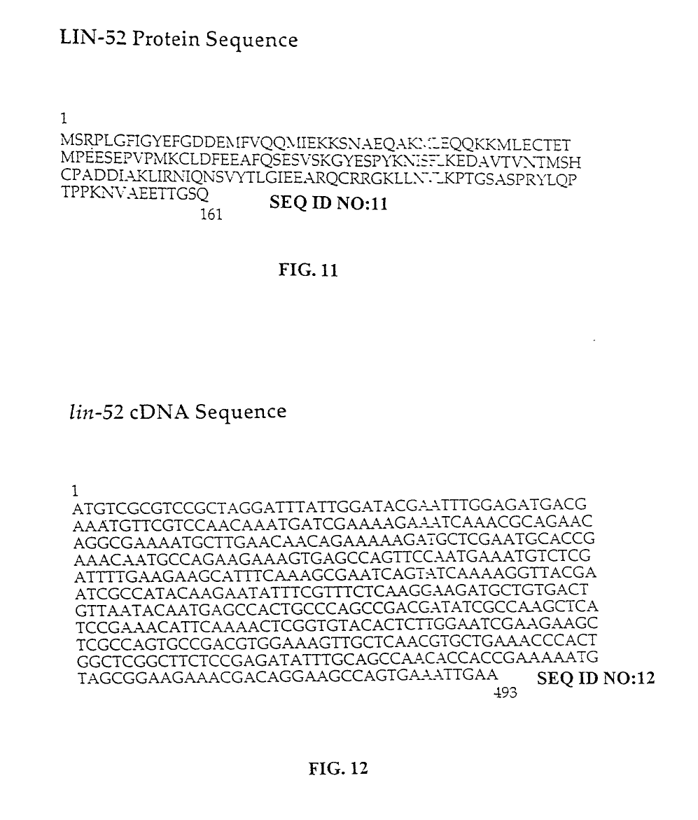

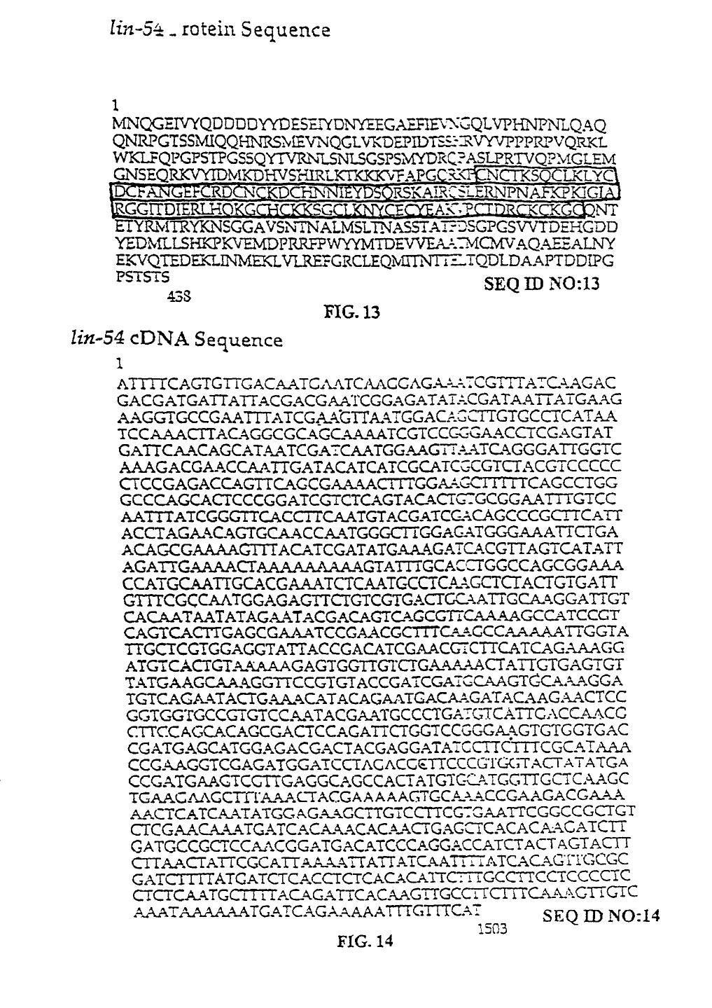

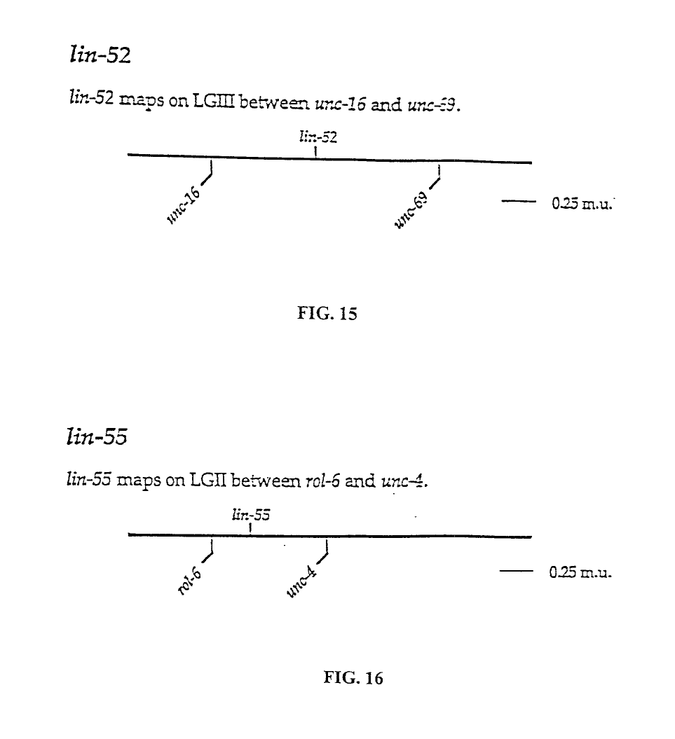

We have also cloned lin-52 (FIGS. 11 and 12) and lin-54 (FIGS. 13 and 14). Like lin-37, these genes do not have homology to known tumor suppressor genes. We searched public sequence databases and found human and mouse cDNA clones that are similar to lin-54. We have isolated more cDNA clones of the human gene and found that its region of similarity extends beyond the sequence found in public databases. Due to the sequence similarity, we speculate that lin-54 shares some common function with the human and mouse genes.

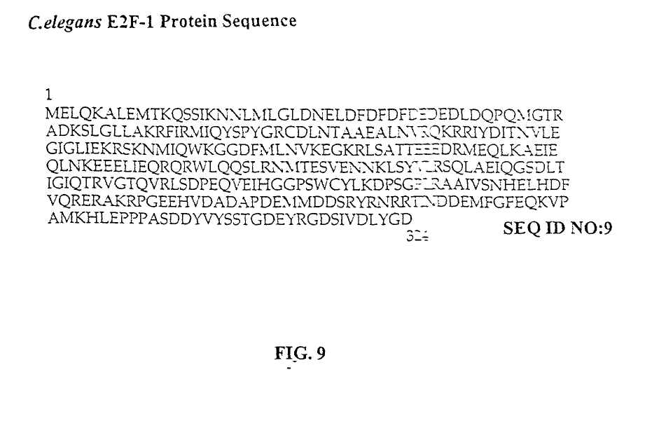

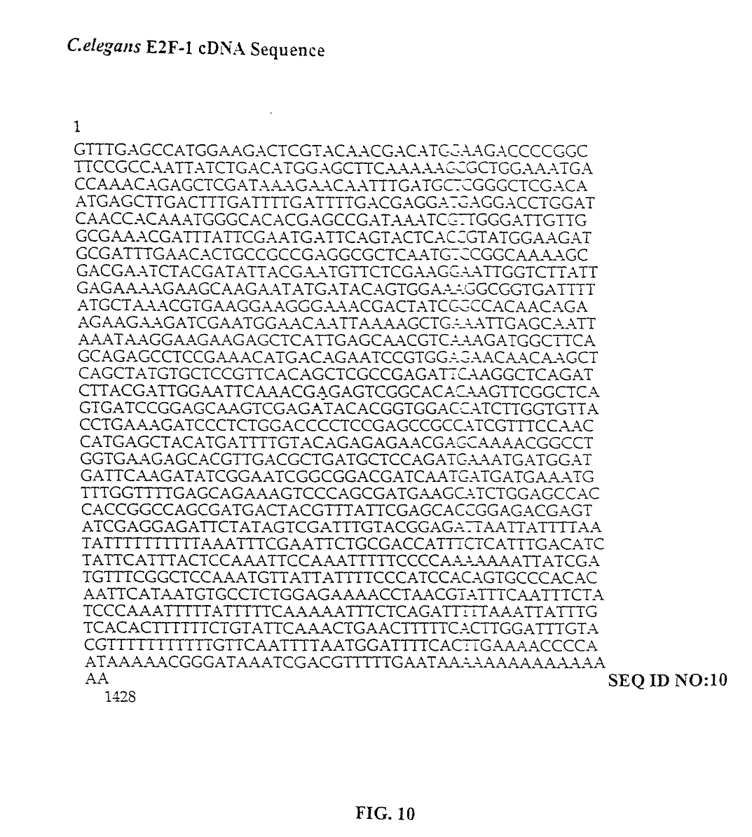

We have also found an E2F-like gene in worms (FIGS. 9 and 10). This gene, which we are referring to as C. elegans E2F-1, was identified because of its similarity to mammalian E2F genes. We have conducted experiments, which are described below, that suggest that C. elegans E2F-1 is a member of the worm synMuv genetic pathway. To summarize, the synMuvs act to negatively regulate vulval induction. At a molecular level, vulval induction is controlled by a Ras pathway. Ras pathway members have been shown to mutate in a variety of human cancers to give an increased level of pathway signalling. We have now shown that a pathway related to the tumor suppressor Rb negatively regulates Ras pathway signalling. We have also identified a number of molecules which act as part of this Rb-related pathway. The striking parallel between the Rb pathway in mammals and the Rb-related pathway we have discovered in worms indicates that further characterization of the synthetic multivulva genes and their mammalian homologs, where appropriate, will provide insight into how cell proliferation is regulated in mammals, particularly humans.

Experiments which stem directly from this research include searches for mammalian homologs of the novel synMuv genes. Such homologs may function in activating, enhancing, or otherwise intensifying the effects of tumor suppressors or oncogenes in mammals.

Genetic enhancer or suppressor screens may be performed to identify new genes which may function in initiating, enhancing, or otherwise interfacing with this Rb-related pathway. In addition, knowing the association provided herein between the synMuv genes and proliferative disease pathways in mammals, one skilled in the art can readily devise drug screens to search for compounds that affect cell proliferation. Specifically, compounds which block the Muv phenotype of synMuv mutant animals are potential antitumor agents. Compounds which stimulate cell division in animals with a single, silent synMuv mutation are likely to be agonists of cell proliferation and may act in a manner analogous to growth factors.

By providing insight regarding the function of the synMuv genes in tumor suppression, we have provided, in concert with generally known molecular biology and nematodes genetic methods, the necessary elements of such methods and the compounds required for the practice of such methods.

II. Model for synMuv Action

FIG. 1 shows the sites of action of the synMuv genes and the lines indicate sites of negative regulation of the Ras pathway that mediates vulval induction.

III. Cloning of lin-52.

lin-52 has been cloned and it encodes a novel predicted protein of amino acids (FIGS. 11 and 12). The n771 allele contains a missense mutation in the gene. Reverse-transcriptase-polymerase chain reaction (RT-PCR) analysis of this gene revealed that its RNA was SL2 trans-spliced. In C. elegans, SL2 trans-splicing is found most often in RNA from genes downstream of operons.

IV. Cloning of lin-55

lin-55 maps on LGII between rol-6 and unc-4. We first obtained cosmid rescue and identified a candidate gene (FIG. 16). We then proceeded to clone the gene. lin-55 encodes a protein similar to DP protein family members (FIG. 8). A single mutant allele of lin-55, n2994, has been identified. Deficiency studies indicate that the allele results in a partial reduction of LIN-55 function.

V. Cloning of lin-37

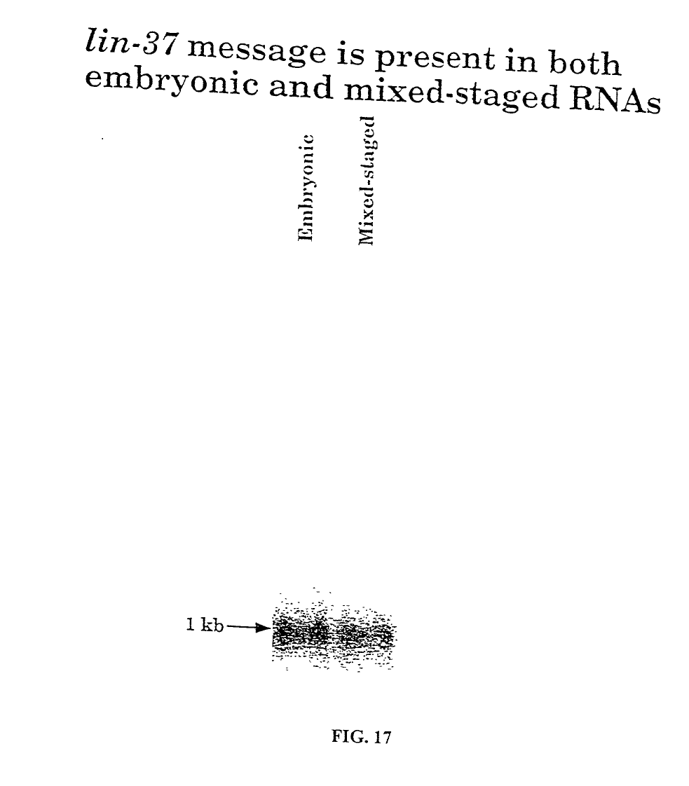

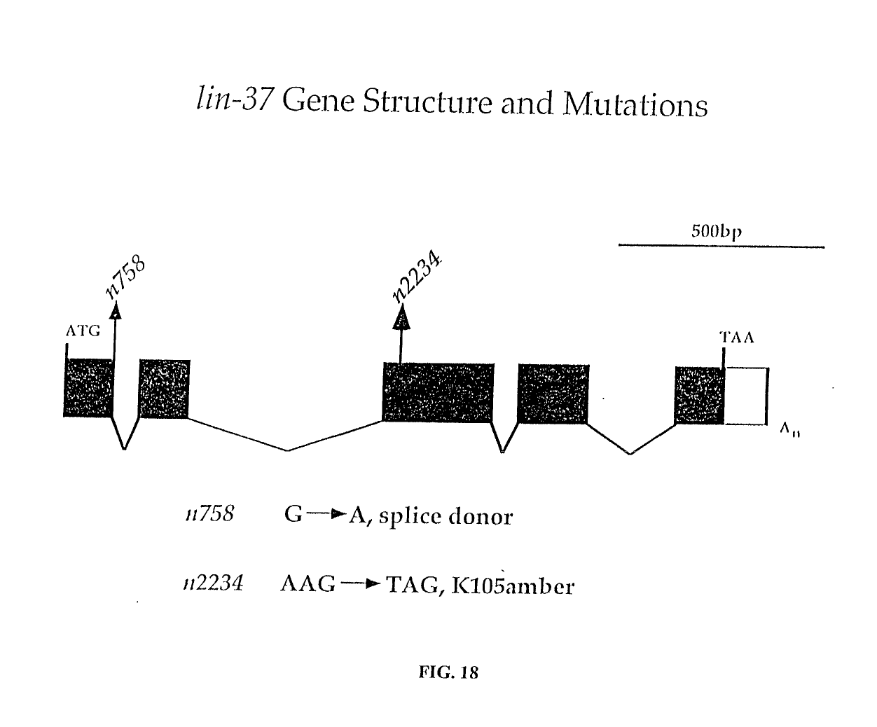

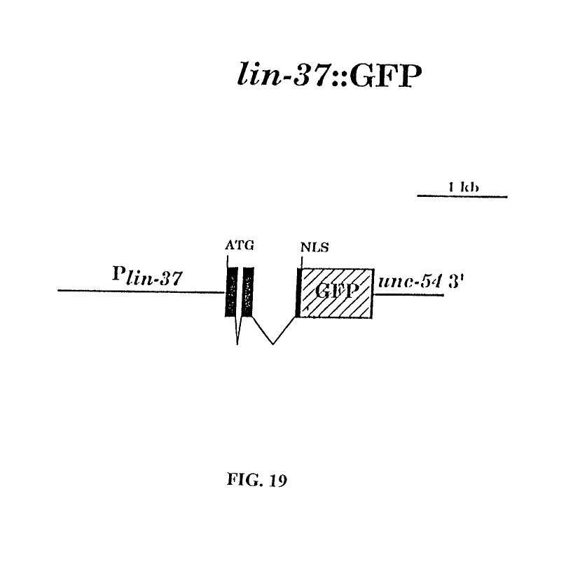

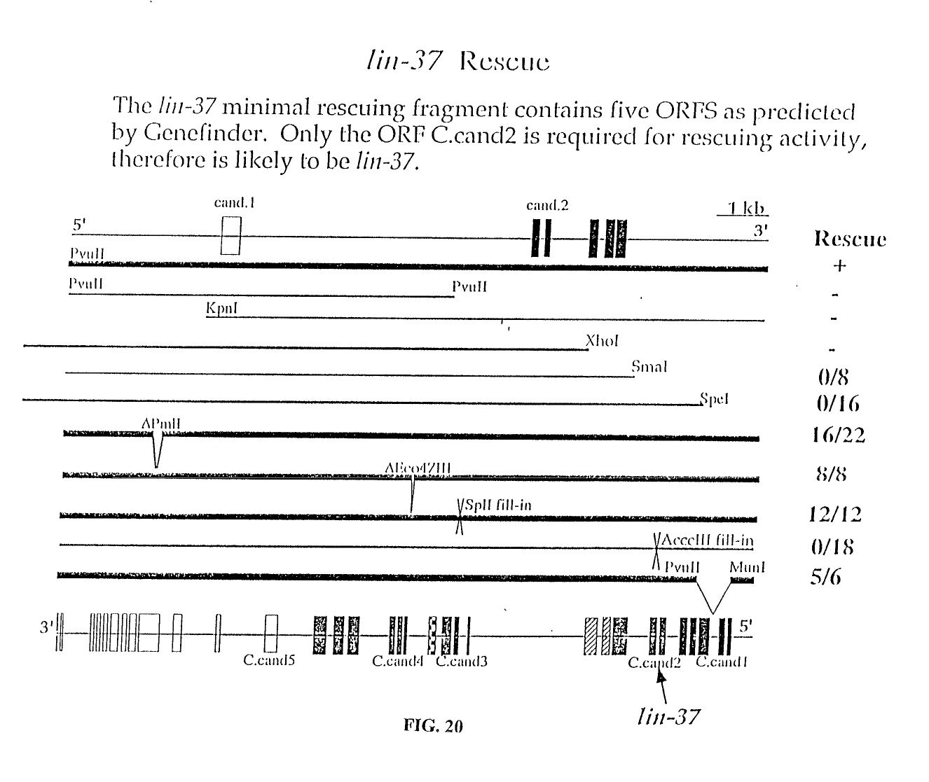

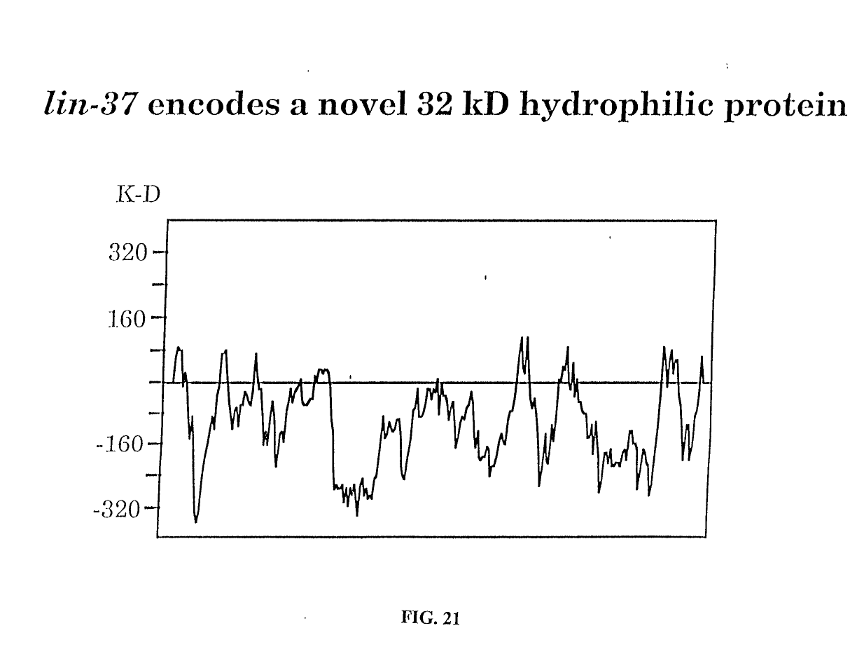

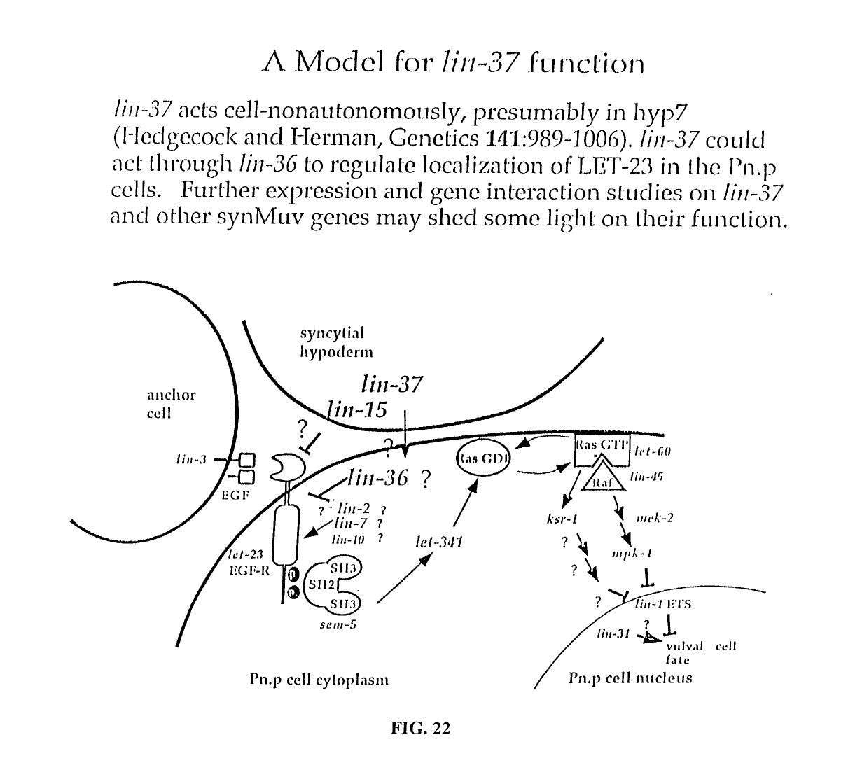

The lin-37 transcript is approximately 1 kb in size (FIG. 3) and is present both in embryonic and mixed-staged RNAs, as revealed by Northern analysis (FIG. 17). A cDNA isolated from the Okkema embryonic cDNA library is about 950 bp in size and can rescue the lin-37 Muv phenotype when expressed under the control of the col-10 promoter (FIG. 20; col-10 encodes a cuticle collagen and is highly expressed in the hypodermis and its precursor cells). The predicted polypeptide product of 31.5 kD is novel (FIG. 21. We determined the sequence of the coding region of the two existing lin-37 alleles (FIG. 18). n758 contains a splice donor mutation in the first intron and is likely to be a null allele. n2234 contains a nonsense mutation in the middle of the coding sequence. A lin-37::gfp transgene was expressed broadly in embryos and in hypodermal cells and vulval cells throughout larval development, consistent with its cell-nonautonomous site of action (FIGS. 19 and 22; Hedgecock and Herman, Genetics 141:989-1006, 1995).

VI. Cloning of lin-35

We mapped lin-35 between unc-40 and the Tc1 polymorphism stP124 on LGI. We identified two overlapping cosmids from this interval, C03E6 and C32F10, each of which rescued the Muv phenotype of a lin-35; lin-15A strain in germline transformation experiments. The smallest subclone that retained rescuing activity was a 9.3 kb XhoI-EcoRV fragment from the region of overlap between C03E6 and C32F10. Using the 9.3 kb minimal rescuing fragment as a probe, we detected a single 3.2 kb transcript in both embryonic and mix-staged poly(A)+RNA on a northern blot, and we isolated cDNA clones from an embryonic cDNA library and determined the complete sequence of the longest cDNA (3.2 kb). We deduced the gene structure by comparing the genomic and the cDNA sequences. The cDNA contains a single open reading frame (ORF) of 961 amino acids and appears to be full-length by three criteria: its size matches that of the transcript detected on the northern blot, it contains the last 11 nucleotides of the SL1 trans-spliced leader sequence at its 5′ end and a poly(A) tail at its 3′ end, and expression of this cDNA under control of the C. elegans heat-shock promoters rescued the lin-35 mutant phenotype. To confirm that the rescuing activity observed was indeed lin-35 activity, we identified the molecular lesions associated with the eight existing lin-35 alleles. Six alleles are nonsense mutations, and two alleles have an identical splice-acceptor mutation in the fourth intron despite their independent isolation. The allele n745 contains an early nonsense mutation predicted to eliminate the C-terminal 84% of LIN-35. This allele may completely eliminate lin-35 function.

The predicted LIN-35 protein shares significant sequence similarity with the mammalian pocket proteins, which include the tumor suppressor Rb, p107 and p130. The similarity extends across the entire lengths of the proteins, including the “A/B pocket” domains, which mediate interactions with proteins important for cell-cycle regulation, such as viral oncoproteins and E2F. Overall, LIN-35 is 20% identical to p130, 19% to p107, 15% to Rb and 16% to RBF, an Rb-related protein in Drosophila melanogaster. Regions important for pocket protein function are more conserved. For example, the N-terminal region of the B pocket domain of LIN-35 (amino acid residues 744-839) is 34% identical to p130, 34% to p107, 29% to Rb and 30% to RBF. The spacer region that separates the A and B pockets of LIN-35 is not related in sequence to those of the pocket proteins. This spacer is short in length, like that of Rb. In contrast, p130 and p107 have much longer spacer regions, which are conserved between them and mediate their stable association with cyclin/cyclin-dependent kinase (CDK) complexes. Because LIN35 is not particularly similar in sequence to any one of the three mammalian pocket proteins, lin-35 may have diverged from an ancestor of the three mammalian genes before these three genes diverged from each other. LIN-35 protein is present in vulval cells.

We raised polyclonal antibodies against a peptide from the N-terminal region of LIN-35. Affinity-purified antisera recognized a single protein of about 110 kDa present in wild-type protein extracts but absent in lin-35(n745) extracts on western blots. Thus, the antisera appeared to recognize specifically the LIN-35 protein product.

We stained wild-type and lin-35 mutant worms with the purified antiLIN-35 peptide antisera. In wild-type animals, the nuclei of most if not all cells in embryos and newly hatched L1 larvae stained. In older larvae and adults, staining appeared diminished and became restricted to the nuclei of certain cells in the head and tail regions and of the P(3-8).p cells and their descendants. We did not observe any staining during these stages in hyp7, the hypodermal syncytium that surrounds the P(3-8).p cells; hyp7 is the proposed site of action of lin-15 and lin37. The presence of LIN-35 protein in the P(3-8).p cell descendants is consistent with the hypothesis that lin-35 acts cell-autonomously to regulate vulval development. The distribution of LIN-35 protein suggests that lin-35 may have other functions. However, mutations in lin-35 do not have any obvious pleiotropic effects. It is possible that other functions of lin-35, like those involved in vulval development, are redundant.

VII. Cloning of lin-53

Based on the finding that lin-35 encodes a protein similar to Rb, we reasoned that other genes in the class B synMuv pathway might also be evolutionarily conserved. We identified from the C. elegans sequence database genomic sequences and expressed sequence tags (ESTs) predicted to encode proteins similar to proteins known to interact with Rb, and we compared their map positions with those of known class B synMuv genes. The cosmid K07A1 contained two predicted genes, named K07A1.12 and K07A1.11, both similar to RbAp48 (p48) and RbAp46 (p46), two closely related proteins that bind to Rb in vivo. This cosmid mapped near the class B synMuv gene lin-53 between unc-29 and lin-11 on LGI. K07A1.11 lies about 100 bp 3′ to K07A1.12 in the same orientation. To investigate whether lin-53 corresponded to either of these genes, we tested to see if cosmid K07A1 would rescue the Muv phenotype of a lin53(n833); lin-15A strain. Since the two existing alleles of lin-53 are semi-dominant (see below), we looked for a partial rescue. Transgenic animals carrying K07A1 exhibited a Muv phenotype of reduced expressivity and penetrance compared to non-transgenic animals, indicating partial rescue.

To confirm that K07A1 indeed contained lin-53 activity, we determined the sequence of K07A1.12 and K07A1.11 from the two independently isolated lin53 alleles, n833 and n2978, and found they carried an identical mutation in K07A1.12 (see below) and had no mutation in K07A1.11. We therefore conclude that K07A1.12 is lin-53. We used EST clones that correspond to each gene as probes to screen for additional cDNAs. The longest lin-53 cDNA contained at its 5′ end the last seven nucleotides from the C. elegans trans-spliced leader SL1 followed by three nucleotides upstream of the first ATG and at its 3′ end a poly(A) tail. The longest K07A1.11 cDNA contained at its 5′ end the last ten nucleotides from the C. elegans trans-spliced leader SL2 followed by six nucleotides upstream of the first ATG and at its 3′ end a poly(A) tail. The tandem arrangement of these two genes in close proximity, with the message of the 3′ gene SL2 trans-spliced, suggests that they form a complex locus and are co-transcribed.

The LIN-53 protein is 72% identical to p48 and 70% to p46, while K07A1.11 is 53% identical to p48 and 52% to p46. p48 and p46 are 7 WD-repeat proteins, which are regulatory proteins that mediate protein-protein interactions. Several p48-related proteins have been identified in different organisms, including the p55 subunit of the Drosophila melanogaster chromatin assembly factor-1 and the Saccharomyces cerevesiae proteins Msi1p and Hat2p. LIN-53 is 72%, 27% and 25% identical to these proteins, respectively. The mutation in lin-53(n833) and lin-53(n2978) animals causes an leucine-to-phenylalanine change at a conserved leucine in the fifth WD domain.

Since K07A1.11 is 54% identical to LIN-53, we tested whether expression of K07A1.11 could rescue the lin-53 mutant phenotype. Driven by the col-10 gene promoter, which is strongly expressed in hypodermal and hypodermal blast cells including the P(3-8).p cells during larval development, expression of the lin-53 cDNA but not of the K07A1.11 cDNA partially rescued the Muv phenotype. This result suggests that K07A1.11 cannot substitute for wild-type lin-53 function.

Wild-type lin-53 activity is required for the class B synMuv pathway To investigate further the role of lin-53 in the class B synMuv pathway, we used RNA-mediated interference (RNAi), which has been shown to produce a specific phenocopy of the loss-of-function phenotype of a targeted gene by an unknown mechanism (Fire et al., Nature 391, 806-811, 1998). We assayed the phenotypes of the progeny of wild-type, lin-15A or lin-15B mothers injected with antisense RNA derived from a lin-53 cDNA clone. In all cases, antisense RNA injection caused embryonic lethality, suggesting that lin-53 is required during embryogenesis. Because of this lethality, we were unable to use the RNAi technique to address the role of lin-53 during vulval induction. Instead, we used the col-10 promoter to drive expression of the antisense strand of a lin-53 cDNA in hypodermal and hypodermal blast cells. About 18% of lin-15A animals carrying the P_{col-10} antisense lin-53 construct were Muv, and this Muv phenotype was dependent on the presence of the lin-15A mutation. Neither antisense expression of the K07A1.11 cDNA nor sense expression of the lin-53 or K07A1.11 cDNAs had any effect in similar experiments. These experiments suggest that wild-type lin-53 activity is required for the class B synMuv pathway.

Unlike other synMuv mutations, lin-53(n833) and lin-53(n2978) cause a semi-dominant class B synMuv phenotype, e.g., lin-53(n833)/+; lin-15A animals have an incompletely penetrant Muv phenotype. Since loss of lin-53 function causes a class B synMuv phenotype, as indicated by our antisense experiments, lin-53 might be a haplo-insufficient locus; alternatively, lin-53(n833) might be a dominant-negative mutation. To distinguish between these two possibilities, we examined the phenotypes of animals of genotype +/Df; lin-15A (the Df chromosome was deleted for the lin-53 locus). Five of 413 +/Df; lin-15A animals examined were Muv. This penetrance of the Muv phenotype is much lower than in a lin-53(n833)/+; lin-15A strain, indicating that a two-fold reduction in wild-type lin-53 activity only occasionally causes a synMuv phenotype and that lin53(n833) is unlikely to simply reduce or eliminate lin-53 function. Rather, lin53(n833) is probably a dominant-negative mutation. Consistent with this hypothesis, expression of a lin-53 cDNA carrying the n833 mutation (L292F) driven by the col-10 promoter caused a partially penetrant Muv phenotype in lin53(+); lin-15A animals. The lin-53(n833) mutation appears to affect only vulval development, while the lin-53 null phenotype may be embryonic lethality. One explanation for the tissue-specific effect of the lin-53(n833) allele could be that vulval development is particularly sensitive to a decreased dosage of wild-type lin53. Alternatively, the dominant-negative mutant LIN-53 protein may be titrating a factor that becomes limiting specifically in vulval tissue.

A transgene containing the lin-53 cDNA tagged with GFP at its N-terminus and under the control of the endogenous lin-53 promoter was capable of partially rescuing the Muv phenotype of lin-53(n833); lin-15A animals. We examined the GFP expression pattern in animals carrying an integrated array of this transgene and observed GFP expression in most if not all nuclei during embryogenesis and in newly hatched L1s. During larval development, we observed fluorescence in many nuclei in the head and tail regions, similar to the LIN-35 staining pattern seen with the anti-LIN-35 peptide antibody. GFP was also present in hypodermal cells throughout development. At the time of vulval induction, GFP was visible in all P(3-8).p cells and persisted until after the P(3-8).p cell divisions and vulval morphogenesis were complete.

VIII. A Histone Deacetylase May Act in the LIN-35 Rb-Mediated Pathway

Based on recent findings that Rb can recruit a histone deacetylase, HDAC1, to repress transcription from target promoters and the fact that p48 has been identified as a subunit of histone deacetylase, we investigated whether several C. elegans histone deacetylase homologs, hda-1, hda-2 and hda-3, might be involved in the synMuv pathway. RNAi of hda-1 caused embryonic lethality, while RNAi of hda-2 and hda-3 produced no obvious phenotypic abnormality in either a lin-15A or lin-15B background. We then tested whether tissue-specific antisense expression of hda-1 driven by the col-10 promoter can cause a synMuv phenotype. Similar to lin-53 antisense expression, hda-1 antisense expression caused a Muv phenotype in a lin-15A background but not in a wild-type background, i.e., a Class B synMuv phenotype. Antisense expression of the closely related hda-3 gene had no effect. This result suggests that the histone deacetylase gene hda-1 activity is required for the lin-35 Rb-mediated synMuv pathway.

IX. LIN-35 Rb, LIN-53 p48, and HDA-1 interact in vitro

To determine whether LIN-35 Rb can directly bind LIN-53 p48 or HDA-1, as predicted by their sequences, we performed GST pull-down experiments. Bacterially-produced glutothione S-transferase (GST) or GST::LIN53 or GST::HDA-1 fusion proteins immobilized on beads were incubated with different in vitro translated ³⁵S-methionine-labeled proteins. GST::LIN-53 interacted with a fragment of LIN-35Rb that contains the entire A/B pocket (LIN35BX) but not with LIN-35 fragments that lack the intact A/B pocket or with control proteins. Interestingly, GST::LIN-53(L292F) and GST::HDA-1 fusion proteins also interacted with the LIN-35BX fragment. It is worth noting that the HDA-1 sequence lacks a recognizable LXCXE motif, which is involved in the interaction between human Rb and HDAC1. Therefore, HDA-1 does not appear to interact with LIN-35 Rb via an LXCXE motif. GST::LIN-53 and GST::LIN53(L292F) fusion proteins also interacted with HDA-1. None of the proteins tested was retained by GST beads. These results indicate that direct physical interactions can occur between any two of the three proteins LIN-35 Rb, LIN-53 p48 and HDA-1. We have not determined whether a ternary complex containing these three proteins can exist.

X. The lin-35 and lin-53 synMuv Phenotypes Require a Functional RTK/Ras Signaling Pathway

To determine how the lin-35 and lin-53 synMuv genes interact with the Ras signaling pathway during vulval development, we analyzed the vulval phenotype of triple mutants carrying either a lin-35 or a lin-53 mutation, a mutation in a class A synMuv gene and a vulvaless (Vul) mutation in a Ras signaling gene. The synMuv phenotype was epistatic to the Vul phenotype caused by a mutation in the lin-3 gene, which encodes the inductive signal. This observation suggests that the lin-35 and lin-53 genes act downstream of or in parallel to lin-3 and that the synMuv phenotype does not require the lin-3 inductive signal.

By contrast, the Vul phenotypes of mutations in let-23 RTK, sem-5, let-60 Ras, lin-45 Raf and mpk-1 genes were epistatic to the synMuv phenotype. These observations suggest that the lin-35 and lin-53 genes act upstream of or in parallel to the RTK/Ras signaling genes. In other words, the expression of vulval cell fates by the P3.p, P4.p and P8.p cells in synMuv mutants requires a functional RTK/Ras signal transduction pathway. These observations concerning lin-35 and lin-53 are equivalent to previous observations concerning other synMuv genes.

Since in synMuv mutants P5.p, P6.p and P7.p generally express normal vulval cell fates, synMuv gene activities are not required in these three cells. To determine whether the synMuv genes can act in the P5.p, P6.p and P7.p cells, we analyzed the phenotype of triple mutants carrying either a lin-35 or a lin-53 mutation, a mutation in a class A synMuv gene and a Vul mutation in lin-2, lin-7 or lin-10 . These three genes act as positive regulators of the LET-23 RTK activity by localizing LET-23 to the basolateral membrane of the P(3-8).p cells, thus allowing better access to the LIN-3 inductive signal from the anchor cell. Mutations in lin-2, lin-7 and lin-10 in general cause P5.p, P6.p and P7.p to adopt the uninduced 3 fate and result in a Vul phenotype. By contrast, in the triple mutants, P5.p, P6.p and P7.p in general adopt a vulval fate. Thus, in these three cells in the absence of receptor localization the activity of RTK/Ras pathway is insufficient to induce vulval cell fates unless synMuv gene activity is eliminated. This observation reveals that synMuv gene activity can function in P5.p, P6.p and P7.p. We conclude that synMuv gene activity acts in all six P(3-8).p cells to antagonize the activity of the RTK/Ras signaling pathway. XI. Identification and functional characterization of C. elegans E2F-1

We have identified a partial C. elegans cDNA clone that shares similarity with E2F family members. We used this clone to obtain full-length cDNA clones of this gene, which we are calling C. elegans E2F-1 (FIG. 10). We have not identified any mutations in C. elegans E2F-1 but we have conducted experiments that suggest it may function in the worm synMuv pathway. Specifically, we have used a technique referred to as “RNA interference” (Fire et al., supra) to assess the loss-of-function phenotype of C. elegans E2F-1. When injected into adult worms, RNA derived from specific gene can inactivate, by an unknown mechanism, the same gene in the progeny of the injected worm. We injected C. elegans E2F-1 RNA into adult worms and found it causes a synMuv phenotype in the progeny of injected worms. One may now screen for a deletion mutation in the C. elegans E2F-1 gene to determine if it also causes a synMuv phenotype.

IX. Cloning of lin-54 and Identification of Human and Mouse lin-54-like Genes

lin-54 was cloned by standard transformation rescue. It encodes a predicted protein of 438 amino acids (FIGS. 13 and 14). The two alleles of this gene, n2231 and n2990, are both missense mutations.

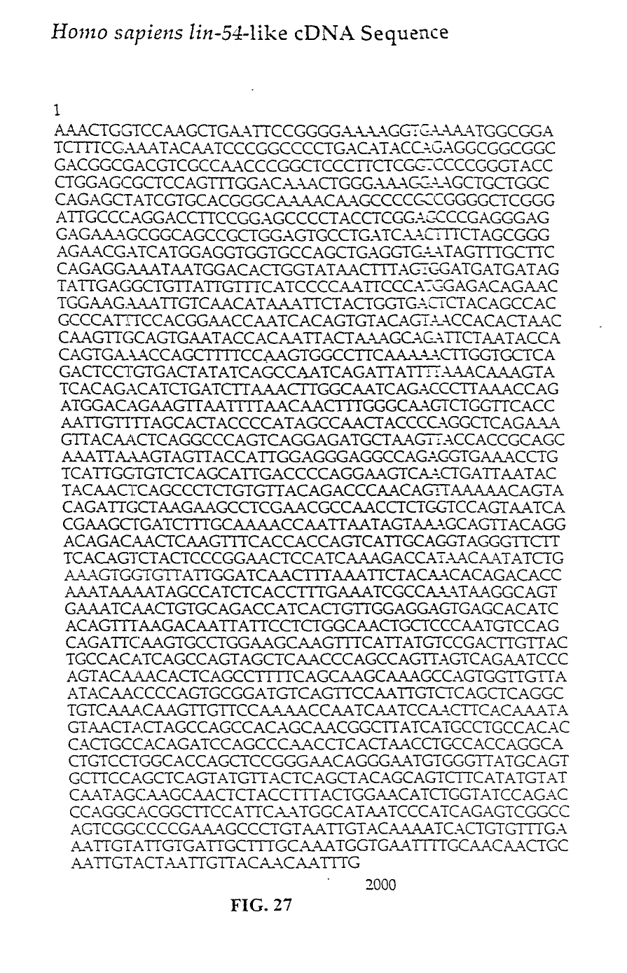

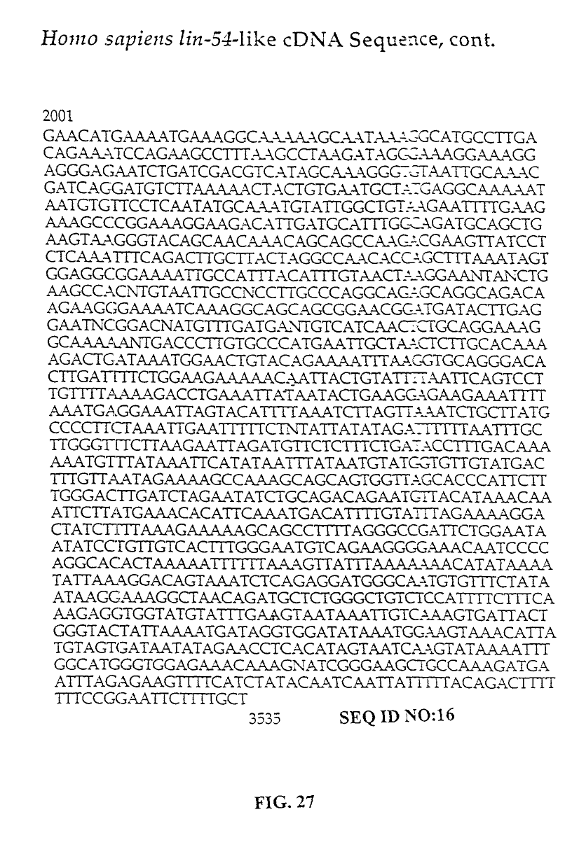

In search of public sequence databases, we found human and mouse cDNA clones that are derived from genes similar to lin-54 (FIGS. 26 and 27). We have subsequently isolated more cDNA clones of the human gene in order to build a complete open reading frame for this gene. Thus far we have an open reading frame that is 3.5 kilobases in length. If translated, the human and mouse genes encode proteins that are similar to LIN-54 in a cysteine-rich domain. Within this domain there appear to be two cysteine repeats with the following signatures, where X denotes an amino acid other than cysteine:

CXCX₄CX₄CXCX₆CX₂CXCX₂C and

CXCX₄CX₄CXCX₆CX₃CXCX₂C

Although the molecular functions of LIN-54 and these human and mouse proteins are unknown, we believe that the high degree of sequence identity in the cysteine-rich domain indicates that the proteins share a common molecular function.

X. Characterization of Interactions Among the synMuv Genes.

Standard yeast two-hybrid techniques are used to characterize the physical interactions between the synMuv gene products. These two-hybrid systems can also be used to detect therapeutic compounds which disrupt the synMuv protein-protein interactions. We have performed two-hybrid analyses with lin-9, lin-15A, lin-15B and lin-36 and found that the LIN-36 protein may self-associate but that none of the other proteins appear to interact with each other. The array in FIG. 23 shows these results.

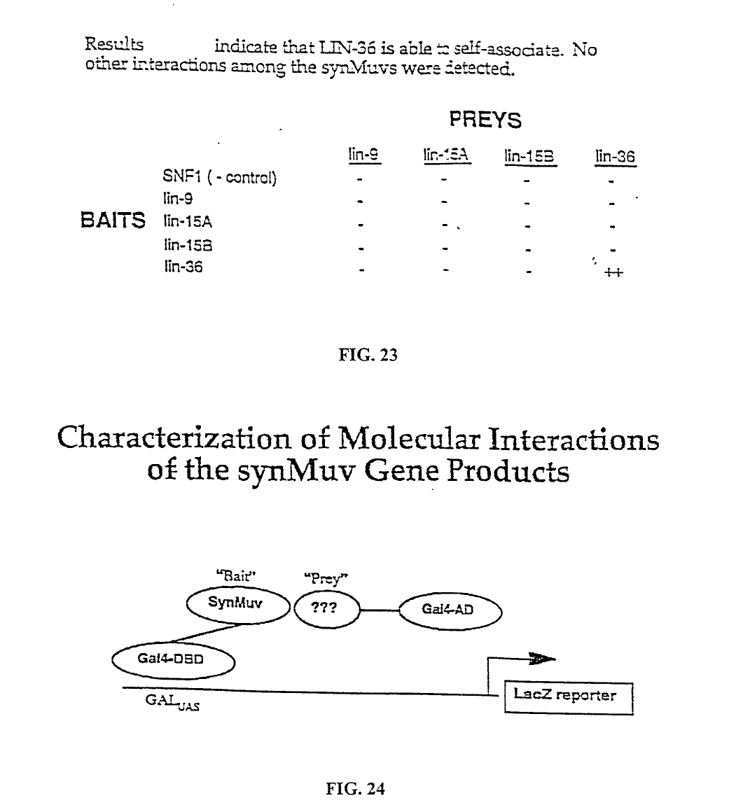

To further study the protein-protein interactions we have constructed the genetic screen shown in FIG. 24. This screen should detect synMuv class B mutations which, in combination with a class A mutation, may be lethal to the animal or may cause the animal to be sterile. Some synMuv B alleles are sterile without a class A mutation in the background. A screen using these mutations would give rise to A⁻, B⁻, Muv animals where the B-mutation confers sterility.

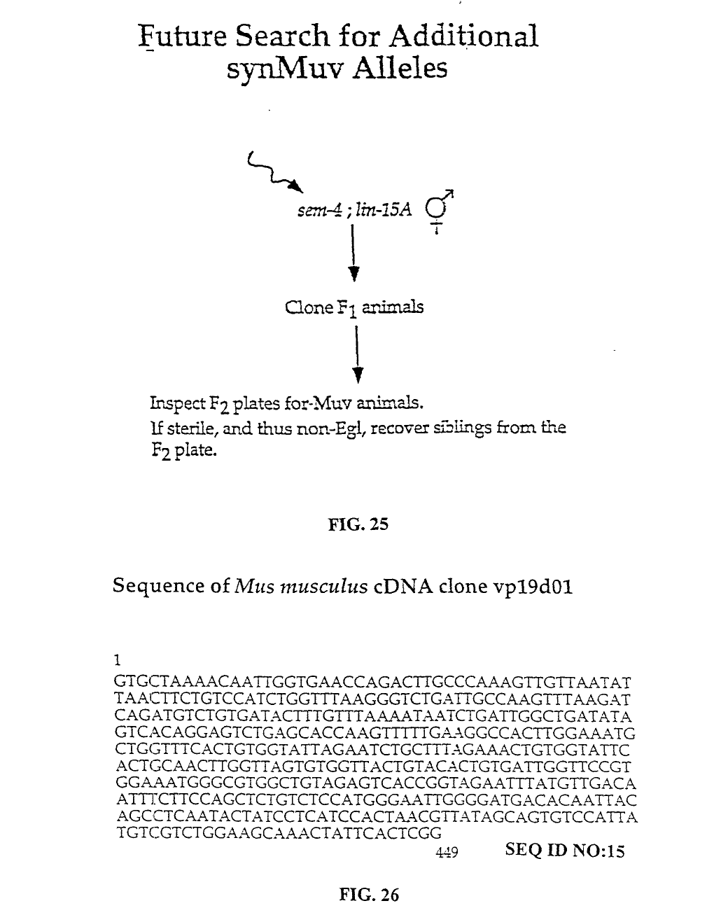

Using this observation we have also constructed a genetic screen based upon the sterility phenotypes to identify more synMuv genes. This strategy is shown in FIG. 25.

Genetic analysis has allowed us to place the class B synMuv genes (lin-9, lin-15B, lin-35, lin-37, lin-51, lin-52, lin-53, lin-54, and lin-55 into a single pathway. Previously, lin-9 and lin-15B had been cloned and sequenced (Beitel, supra; Clark; supra; Huang, supra). The polypeptides encoded by these two genes, however, were novel and contained no known motifs which would allow speculation as to their respective functions.

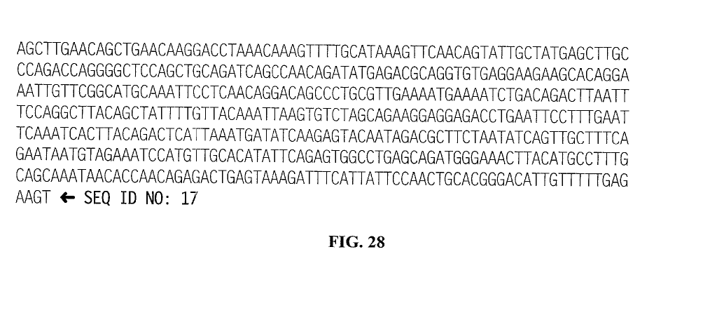

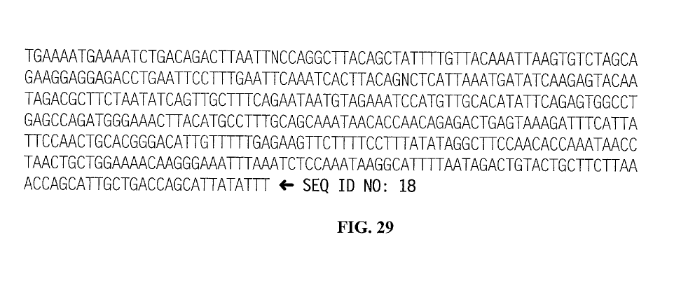

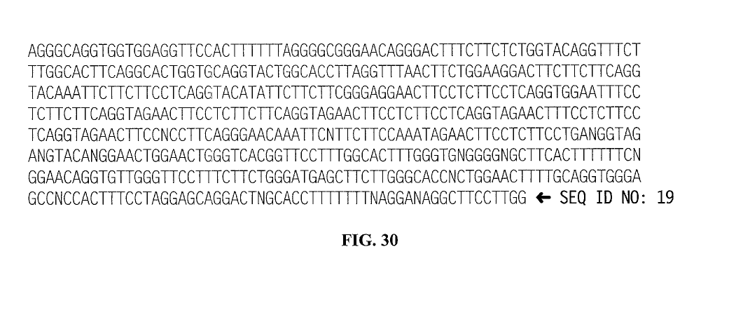

The invention described herein provides the identity of class B synMuv genes lin-35, lin-37, lin-52, lin-53, lin-54, and lin-55. Three of these genes are related to genes in the Rb tumor suppressor pathway. From this discovery, we conclude that the class B synMuv genes, including lin-9 and lin-15B, encode a tumor suppressor pathway. It is likely that C. elegans synMuv genes have ortholog counterparts in vertebrates. One skilled in the art will recognize that these orthologs can be identified using standard techniques in molecular biology, such as screening of cDNA or genomic libraries, degenerate PCR, and the like, described in, for example, Ausubel et al., supra. Orthologs can also be identified using computer-based search programs such as BLAST and dbEST to isolate expressed sequence tags (ESTs) with regions of similarity or identity to a synMuv gene. Using such an approach, we have identified possible vertebrate orthologs of lin-9 and lin-15B (FIGS. 28, 29, and 30). For example, an EST, zr79e11.r1 (Genbank accession number AA256253; SEQ ID NO: 17), from a library made from pooled tissue from human melanocyte, fetal heart, and pregnant uterus, produced the highest score (E=2e−7) when LIN-9 was used to search the EST database translated in all six frames (default settings). There was 32% identity and 61% similarity between LIN-9 and zr79e11.r1 in a stretch of 88 amino acids. A second EST, EST180962 (Genbank accession number AA310125; SEQ ID NO: 18), from a human Jurkat T-cell library, also showed high similarity (58%) in the same region of LIN-9. Similarly, an EST, zp44h06.s1 (Genbank accession number AA211293; SEQ ID NO: 19), from a library made from human muscle, produced the highest score (E=0. 15) when LIN-15B was used to search the EST database translated in all six frames (default settings). There was 39% identity and 62% similarity between LIN-15B and zp44h06.s1 over a stretch of 46 amino acids. The identities included nine conserved proline residues throughout the 46 amino acids. Zp44h06.s1, in turn, has high similarity to the cytoskeletal protein Titin.

Vertebrate counterparts of C. elegans synMuv genes, such as lin-9, lin-15B, and lin-54, are candidate tumor suppressor genes in the Rb pathway. Thus, one can screen for mutations in the human homologs of these genes in patients diagnosed with cancer or in immortalized cell lines. Similarly, the proteins encoded by these genes are candidate targets for anti-cancer drugs. A drug which increases synMuv protein activity may decrease proliferation of tumor cells. In addition, proteins which interact with synMuv proteins or which regulate synMuv gene expression are also candidate tumor suppressors; these proteins can be isolated using standard techniques as described herein or, for example, in Ausubel et al., supra.

XI. synMuv Protein Expression

synMuv genes may be expressed in both prokaryotic and eukaryotic cell types. For those synMuvs which modulate cell proliferation it may be desirable to express the protein under control of an inducible promotor for the purposes of protein production.

In general, synMuv proteins according to the invention may be produced by transformation of a suitable host cell with all or part of a synMuv-encoding cDNA fragment (e.g., the cDNAs described above) in a suitable expression vehicle.

Those skilled in the field of molecular biology will understand that any of a wide variety of expression systems may be used to provide the recombinant protein. The precise host cell used is not critical to the invention. The synMuv protein may be produced in a prokaryotic host (e.g., E. coli) or in a eukaryotic host (e.g., nematodes, Saccharamyces cerevisiae, insect cells, e.g., Sf21 cells, or mammalian cells, e.g., COS 1, NIH 3T3, or HeLa cells). Such cells are available from a wide range of sources (e.g., the American Type Culture Collection, Rockland, Md.; also, see, e.g., Ausubel et al., Current Protocols in Molecular Biology, John Wiley & Sons, New York, 1994). The method of transformation or transfection and the choice of expression vehicle will depend on the host system selected. Transformation and transfection methods are described, e.g., in Ausubel et al., (supra); expression vehicles may be chosen from those provided, e.g., in Cloning Vectors: A Laboratory Manual (P. H. Pouwels et al., 1985, Supp. 1987).

One preferred expression system is the baculovirus system (using, for example, the vector pBacPAK9) available from Clontech (Palo Alto, Calif.). If desired, this system may be used in conjunction with other protein expression techniques, for example, the myc tag approach described by Evan et al. (Mol. Cell Biol. 5:3610-3616, 1985).

Alternatively, a synMuv protein is produced by a stable-transfected mammalian cell line. A number of vectors suitable for stable transfection of mammalian cells are available to the public, e.g., see Pouwels et al. (supra); methods for constructing such cell lines are also publicly available, e.g., in Ausubel et al. (supra). In one example, cDNA encoding the synMuv protein is cloned into an expression vector which includes the dihydrofolate reductase (DHFR) gene. Integration of the plasmid and, therefore, the synMuv protein-encoding gene into the host cell chromosome is selected for by inclusion of 0.01-300 μM methotrexate in the cell culture medium (as described in Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra). Any of the host cells described above or, preferably, a DHFR-deficient CHO cell line (e.g., CHO DHFR⁻ cells, ATCC Accession No. CRL 9096) are among the host cells preferred for DHFR selection of a stable-transfected cell line or DHFR-mediated gene amplification.

Once the recombinant synMuv protein is expressed, it is isolated, e.g., using affinity chromatography. In one example, an anti-synMuv protein antibody (e.g., produced as described herein) may be attached to a column and used to isolate the synMuv protein. Lysis and fractionation of synMuv protein-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short synMuv protein fragments, can also be produced by chemical synthesis (e.g., by the methods described in Solid Phase Peptide Synthesis, 2nd ed., 1984, The Pierce Chemical Co., Rockford, Ill.).

These general techniques of polypeptide expression and purification can also be used to produce and isolate useful synMuv fragments or analogs (described herein).

XI. Anti-synMuv Antibodies

To generate synMuv-specific antibodies, a synMuv coding sequence can be expressed as a C-terminal fusion with glutathione S-transferase (GST) (Smith et al., Gene 67:31-40, 1988). The fusion protein can be purified on glutathione-Sepharose beads, eluted with glutathione cleaved with thrombin (at the engineered cleavage site), and purified to the degree necessary for immunization of rabbits. Primary immunizations can be carried out with Freund's complete adjuvant and subsequent immunizations with Freund's incomplete adjuvant. Antibody titres are monitored by Western blot and immunoprecipitation analyses using the thrombin-cleaved synMuv protein fragment of the GST-synMuv fusion protein. Immune sera are affinity purified using CNBr-Sepharose-coupled synMuv protein. Antiserum specificity is determined using a panel of unrelated GST proteins (including GSTp53, Rb, HPV-16 E6, and E6-AP) and GST-trypsin (which was generated by PCR using known sequences).

As an alternate or adjunct immunogen to GST fusion proteins, peptides corresponding to relatively unique regions of synMuv may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using synMuv expressed as a GST fusion protein.

Alternatively, monoclonal antibodies may be prepared using the synMuv proteins described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specific synMuv recognition by Western blot or immunoprecipitation analysis (by the methods described in Ausubel et al., supra). Antibodies which specifically recognize synMuv are considered to be useful in the invention; such antibodies may be used, e.g., in an immunoassay to monitor the level of synMuv produced by a animal (for example, to determine the amount or subcellular location of synMuv).

Preferably, antibodies of the invention are produced using fragments of the synMuv protein which lie outside highly conserved regions and appear likely to be antigenic, by criteria such as those provided by the Peptidestructure program of the Genetics Computer Group Sequence Analysis Package (Program Manual for the GCG Package, Version 7, 1991) using the algorithm of Jameson and Wolf (CABIOS 4:181, 1988). In one specific example, such fragments are generated by standard techniques of PCR and cloned into the pGEX expression vector (Ausubel et al., supra). Fusion proteins are expressed in E. Coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

XII. Identification of Molecules that Modulate synMuv Protein Expression

Isolation of the synMuv cDNAs also facilitates the identification of molecules which increase or decrease synMuv expression. According to one approach, candidate molecules are added at varying concentrations to the culture medium of cells or nematodes expressing synMuv mRNA. synMuv expression is then measured, for example, by standard Northern blot analysis (Ausubel et al., supra) using a synMuv cDNA (or cDNA fragment) as a hybridization probe (see also Table III). The level of synMuv expression in the presence of the candidate molecule is compared to the level measured for the same cells in the same culture medium but in the absence of the candidate molecule. When nematodes are being used, use of the phenotypes associated with the synMuv pathway may be used as the primary screen for alteration in protein expression.

If desired, the effect of candidate modulators on expression may, in the alternative, be measured at the level of synMuv protein production using the same general approach and standard immunological detection techniques, such as Western blotting or immunoprecipitation with a synMuv-specific antibody (for example, the synMuv antibody described herein).

Candidate modulators may be purified (or substantially purified) molecules or may be one component of a mixture of compounds (e.g., an extract or supernatant obtained from cells; Ausubel et al., supra). In a mixed compound assay, synMuv expression is tested against progressively smaller subsets of the candidate compound pool (e.g., produced by standard purification techniques, e.g., HPLC or FPLC) until a single compound or minimal compound mixture is demonstrated to modulate synMuv expression.

Alternatively, or in addition, candidate compounds may be screened for those which modulate synMuv cell death activity. In this approach, the degree of cell proliferation or the synMuv phenotype in the presence of a candidate compound is compared to the degree of cell death in its absence, under equivalent conditions. Again, such a screen may begin with a pool of candidate compounds, from which one or more useful modulator compounds are isolated in a step-wise fashion. Cell death activity may be measured by any standard assay.

Candidate synMuv modulators include peptide as well as non-peptide molecules (e.g., peptide or non-peptide molecules found, e.g., in a cell extract, mammalian serum, or growth medium on which mammalian cells have been cultured).

Modulators found to be effective at the level of synMuv expression or activity may be confirmed as useful in animal models and, if successful, may be used as anti-cancer therapeutics for either the inhibition of cell death.

XIII. synMuv Therapy

Because expression levels of synMuv genes correlates with the levels of cell death, the synMuv gene also finds use in gene therapy to modulate cell proliferation.

Retroviral vectors, adenoviral vectors, adeno-associated viral vectors, or other viral vectors with the appropriate tropism for cells likely to be involved in the cell proliferation disease may be used as a gene transfer delivery system for a therapeutic synMuv gene construct. Numerous vectors useful for this purpose are generally known (Miller, Human Gene Therapy 5-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, BioTechniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches may also be employed for the introduction of therapeutic DNA into cells otherwise predicted to undergo insufficient or excess cell proliferation. For example, synMuv may be introduced into a cell by the techniques of lipofection (Felgner et al., Proc. Natl. Acad. Sci. USA 84:7413, 1987; Ono et al., Neuroscience Lett 117:259, 1990; Brigham et al., Am. J. Med. Sci. 298:278, 1989; Staubinger and Papahadjopoulos, Meth. Enz. 101:512, 1983); asialorosonucoid-polylysine conjugation (Wu and Wu, J. Biol. Chem. 263:14621, 1988; Wu et al., J. Biol. Chem. 264:16985, 1989); or, less preferably, microinjection under surgical conditions (Wolff et al., Science 247:1465, 1990).

For any of the above approaches, the therapeutic synMuv DNA construct is preferably applied to the site of the predicted cell proliferation event (for example, by injection), but may also be applied to tissue in the vicinity of the predicted event or even to a blood vessel supplying the cells predicted to undergo insufficient or excess cell proliferation.

In the gene therapy constructs, synMuv cDNA expression is directed from any suitable promoter (e.g., the human cytomegalovirus, simian virus 40, or metallothionein promoters), and its production is regulated by any desired regulatory element. For example, if desired, enhancers known to direct preferential gene expression in a particular cell may be used to direct synMuv expression. Such enhancers include, without limitation, those enhancers which are characterized as tissue or cell specific in their expression.

Alternatively, if a synMuv genomic clone is utilized as a therapeutic construct (for example, following its isolation by hybridization with the synMuv cDNA described above), synMuv expression is regulated by its cognate regulatory sequences or, if desired, by regulatory sequences derived from a heterologous source, e.g., any of the promoters or regulatory elements described above.

Less preferably, synMuv gene therapy is accomplished by direct administration of the synMuv mRNA to a cell predicted to undergo excess or insufficient cell proliferation. This mRNA may be produced and isolated by any standard technique, but is most readily produced by in vitro transcription using a synMuv cDNA under the control of a high efficiency promoter (e.g., the T7 promoter). Administration of synMuv mRNA to malignant cells is carried out by any of the methods for direct nucleic acid administration described above.

Ideally, the production of synMuv protein by any gene therapy approach described above results in a cellular level of synMuv that is at least equivalent to the normal, cellular level of synMuv in an unaffected individual. Treatment by any synMuv-mediated gene therapy approach may be combined with more traditional therapies.

Another therapeutic approach included within the invention involves direct administration of recombinant synMuv protein, either to the site of a predicted or desirable cell proliferation event (for example, by injection) or systemically by any conventional recombinant protein administration technique. The actual dosage of synMuv depends on a number of factors, including the size and health of the individual patient, but, generally, between 0.1 mg and 100 mg inclusive are administered per day to an adult in any pharmaceutically-acceptable formulation.

XIV. Administration of synMuv Polypeptides, synMuv Genes or Modulators of synMuv Synthesis or Function

A synMuv protein, gene, or modulator may be administered with a pharmaceutically-acceptable diluent, carrier, or excipient, in unit dosage form. Conventional pharmaceutical practice may be employed to provide suitable formulations or compositions to administer synMuv to patients suffering from or presymptomatic for a synMuv-associated carcinoma. Any appropriate route of administration may be employed, for example, parenteral, intravenous, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for synMuv modulatory compounds include ethylenevinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a synMuv protein, gene, or modulatory compound may be combined with more traditional therapies for the disease such as surgery, radiation, or chemotherapy for cancers.

XV. Detection of A Condition Involving Altered Cell Proliferation

synMuv polypeptides and nucleic acid sequences find diagnostic use in the detection or monitoring of conditions involving aberrant levels of cell proliferation. A decrease or increase in the level of synMuv production may provide an indication of a deleterious condition. Levels of synMuv expression may be assayed by any standard technique. For example, its expression in a biological sample (e.g., a biopsy) may be monitored by standard Northern blot analysis or may be aided by PCR (see, e.g., Ausubel et al., supra; PCR Technology: Principles and Applications for DNA Amplification, ed., H. A. Ehrlich, Stockton Press, NY; and Yap and McGee, Nucl. Acids. Res. 19:4294, 1991).

Alternatively, a patient sample may be analyzed for one or more mutations in the synMuv sequences using a mismatch detection approach. Generally, these techniques involve PCR amplification of nucleic acid from the patient sample, followed by identification of the mutation (i.e., mismatch) by either altered hybridization, aberrant electrophoretic gel migration, binding or cleavage mediated by mismatch binding proteins, or direct nucleic acid sequencing. Any of these techniques may be used to facilitate mutant synMuv detection, and each is well known in the art (see, for example, Orita et al., Proc. Natl. Acad. Sci. USA 86:2766-2770, 1989; and Sheffield et al., Proc. Natl. Acad. Sci. USA 86:232-236, 1989).

In yet another approach, immunoassays are used to detect or monitor synMuv protein in a biological sample. synMuv-specific polyclonal or monoclonal antibodies (produced as described above) may be used in any standard immunoassay format (e.g., ELISA, Western blot, or RIA assay) to measure synMuv polypeptide levels; again comparison is to wild-type synMuv levels, and a decrease in synMuv production is indicative of a condition involving altered cell proliferation. Examples of immunoassays are described, e.g., in Ausubel et al., supra. Immunohistochemical techniques may also be utilized for synMuv detection. For example, a tissue sample may be obtained from a patient, and a section stained for the presence of synMuv using an anti-synMuv antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (supra).

In one preferred example, a combined diagnostic method may be employed that begins with an evaluation of synMuv protein production (for example, by immunological techniques or the protein truncation test (Hogerrorst et al., Nature Genetics 10:208-212, 1995) and also includes a nucleic acid-based detection technique designed to identify more subtle synMuv mutations (for example, point mutations). As described above, a number of mismatch detection assays are available to those skilled in the art, and any preferred technique may be used (see above). By this approach, mutations in synMuv may be detected that either result in loss of synMuv expression or loss of synMuv biological activity.

Mismatch detection assays also provide the opportunity to diagnose a synMuv-mediated predisposition to diseases of cell proliferation. For example, a patient heterozygous for a synMuv mutation may show no clinical symptoms and yet possess a higher than normal probability of developing one or more types of diseases. Given this diagnosis, a patient may take precautions to minimize their exposure to adverse environmental factors (for example, UV exposure or chemical mutagens) and to carefully monitor their medical condition (for example, through frequent physical examinations). This type of synMuv diagnostic approach may also be used to detect synMuv mutations in prenatal screens.

The synMuv diagnostic assays described above may be carried out using any biological sample (for example, any biopsy sample or bodily fluid or tissue) in which synMuv is normally expressed. Identification of a mutant synMuv gene may also be assayed using these sources for test samples. Alternatively, a synMuv mutation, particularly as part of a diagnosis for predisposition to synMuv-associated proliferative disease, may be tested using a DNA sample from any cell, for example, by mismatch detection techniques; preferably, the DNA sample is subjected to PCR amplification prior to analysis.

### OTHER EMBODIMENTS

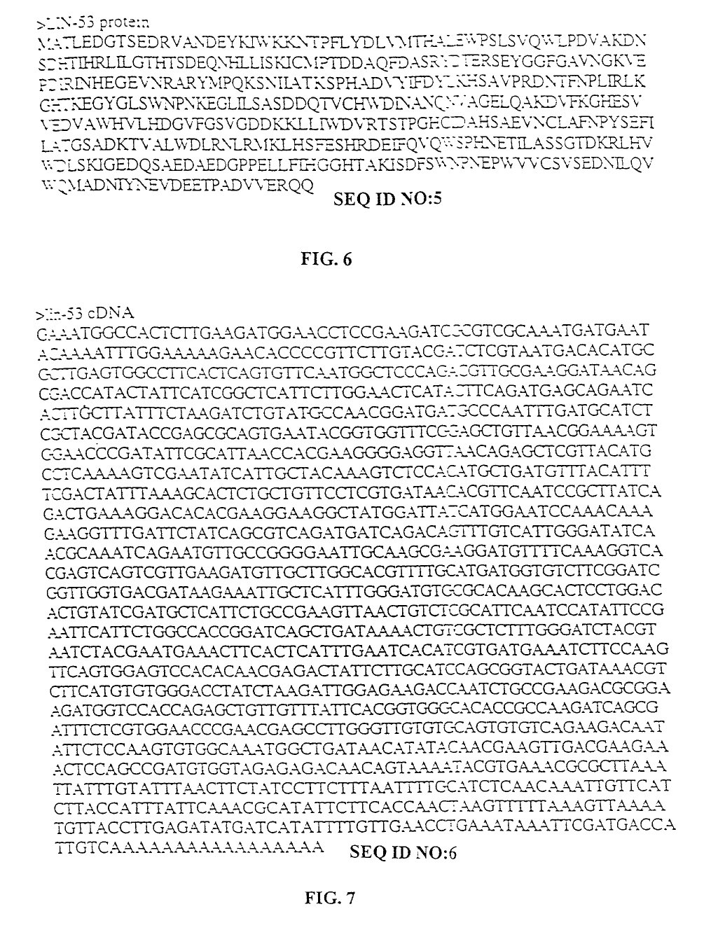

In other embodiments, the invention includes any protein which is substantially identical to a synMuv polypeptide (FIGS. 2, 4, 6, 8, 9, 11, 13), or encoded by a nucleic acid of FIGS. 26 or 27); such homologs include other substantially pure naturally-occurring mammalian synMuv proteins as well as allelic variants; natural mutants; induced mutants; DNA sequences which encode proteins and also hybridize to the synMuv DNA sequences of FIGS. 3, 5, 7, 8, 10, 12, 14, 26, or 27, under high stringency conditions or, less preferably, under low stringency conditions (e.g., washing at 2× SSC at 40° C. with a probe length of at least 40 nucleotides); and proteins specifically bound by antisera directed to a synMuv polypeptide. The term also includes chimeric polypeptides that include a synMuv portion.

The invention further includes analogs of any naturally-occurring synMuv polypeptide. Analogs can differ from the naturally-occurring synMuv protein by amino acid sequence differences, by post-translational modifications, or by both. Analogs of the invention will generally exhibit at least 85%, more preferably 90%, and most preferably 95% or even 99% identity with all or part of a naturally-occurring synMuv amino acid sequence. The length of sequence comparison is at least 15 amino acid residues, preferably at least 25 amino acid residues, and more preferably more than 35 amino acid residues. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring synMuv polypeptide by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes synMuv polypeptide fragments. As used herein, the term “fragment,” means at least 20 contiguous amino acids, preferably at least 30 contiguous amino acids, more preferably at least 50 contiguous amino acids, and most preferably at least 60 to 80 or more contiguous amino acids. Fragments of synMuv polypeptides can be generated by methods known to those skilled in the art or may result from normal protein processing (e.g., removal of amino acids from the nascent polypeptide that are not required for biological activity or removal of amino acids by alternative mRNA splicing or alternative protein processing events).

Preferable fragments or analogs according to the invention are those which facilitate specific detection of a synMuv nucleic acid or amino acid sequence in a sample to be diagnosed.

All publications and patent applications mentioned in this specification are herein incorporated by reference to the same extent as if each independent publication or patent application was specifically and individually indicated to be incorporated by reference.

What is claimed is:

1. A purified nucleic acid comprising a nucleotide sequence that hybridizes under high stringency conditions to a probe comprising at least 75 nucleotides that are complementary to a synMuv gene.

2. The nucleic acid of claim 1, wherein said synMuv gene is a C. elegans synMuv gene, and wherein said C. elegans synMuv gene comprises a sequence chosen from SEQ ID NOS:2, 4, 6, 8, 10, 12, 14, 15, and 16.

3. A substantially pure DNA encoding a synMuv polypeptide selected from the group consisting of LIN-37, LIN-35, LIN-55, LIN-53, LIN-52, LIN-54, and E2F-1.

4. A substantially pure nucleic acid encoding an amino acid sequence selected from the group consisting of SEQ ID NOS:1, 3, 5, 7, 9, 11, and 13.

5. A substantially pure nucleic having the sequence of SEQ ID NO:15, or degenerate variants thereof.

6. A vector comprising the nucleic acid of claim 3, said vector being capable of directing expression of the peptide encoded by said nucleic acid in a vector-containing cell.

7. A cell which contains nucleic acid encoding a synMuv polypeptide selected from the group consisting of LIN-37, LIN-35, LIN-55, LIN-53, LIN-52, LIN-54, and E2F-1, said nucleic acid being a nucleic acid which does not naturally occur in said cell in the position in which it is present.

8. A purified nucleic acid comprising a sequence which hybridizes under high stringency conditions to at least a portion of a sequence chosen from SEQ ID NOS:17, 18, and 19, said portion being at least 50 nucleotides, and said polypeptide having at least one synMuv biological activity.

9. The purified nucleic acid of claim 8, wherein said nucleic acid comprises at least a portion of a sequence chosen from SEQ ID NOS: 17, 18, or 19.

10. A transgenic cell which contains the nucleic acid encoding a synMuv polypeptide selected from the group consisting of LIN-37, LIN-35, LIN-55, LIN-53, LIN-52, LIN-54, and E2F-1, said nucleic acid being a nucleic acid which does not naturally occur in said cell in the position in which it is present.

11. A substantially pure synMuv polypeptide.

12. The polypeptide of claim 11, wherein said polypeptide is selected from the group consisting of SEQ ID NOS:1, 3, 5, 7, 9, 11, and 13.

13. The polypeptide of claim 11, wherein said synMuv polypeptide is a mammalian synMuv polypeptide.

14. The polypeptide of claim 11, wherein said polypeptide is LIN-54 polypeptide.

15. The polypeptide of claim 11, wherein said polypeptide is encoded by DNA which hybridizes at high stringency to at least a portion of a sequence chosen from SEQ ID NOS:17, 18, and 19, said portion being at least 50 nucleotides, and said polypeptide having at least one synMuv biological activity.

16. A method of modulating cell proliferation of a cell, said method comprising administering to said cell a proliferation modulating amount of synMuv polypeptide.

17. The method of claim 16, wherein said cell is in a mammal.

18. The method of claim 16, wherein said mammal is a human.

19. A synMuv gene isolated according to the method comprising:
(a) providing a cell sample;
(b) introducing by transformation into said cell sample a candidate synMuv gene;
(c) expressing said candidate synMuv gene within said cell sample; and
(d) determining whether said cell sample exhibits an altered cell proliferation response, whereby an altered level of cell proliferation identifies a synMuv gene.

20. A purified antibody which binds specifically to a synMuv polypeptide.

21. A method of identifying a compound which modulates cell proliferation, said method comprising:
(a) providing a cell expressing a gene operably linked to a synMuv gene promoter;
(b) contacting said cell with a candidate compound; and
(c) monitoring the expression of said gene, an alteration in the level of expression of said gene indicating the presence of a compound which modulates cell proliferation.

22. The method of claim 21, wherein said gene is a synMuv gene which hybridizes at high stringency to at least a portion of a sequence chosen from SEQ ID NOS:17, 18, and 19, said portion being at least 50 nucleotides, and said polypeptide having at least one synMuv biological activity.

23. The method of claim 21, wherein said gene expression is measured by assaying the protein level of the expressed gene.

24. The method of claim 21, wherein said gene expression is measured by assaying the RNA level of the expressed gene.

25. A method of identifying a synMuv-binding polypeptide, said method comprising:
(a) providing a synMuv polypeptide;
(b) contacting said synMuv polypeptide with a candidate polypeptide; and
(c) monitoring the binding of said candidate polypeptide to said synMuv polypeptide, said binding indicating the presence of a synMuv-binding polypeptide.

26. A method of diagnosing an animal for the presence of an cell proliferation disease or an increased likelihood of developing a cell proliferation disease, said method comprising:
(a) isolating a sample of nucleic acid from said animal; and
(b) determining whether said nucleic acid comprises a mutated synMuv gene,
a mutation in said nucleic acid being an indication that said animal has an cell proliferation disease or an increased likelihood of developing a cell proliferation disease.

27. A method of diagnosing an animal for the presence of a cell proliferation disease or an increased likelihood of developing a cell proliferation disease, said method comprising measuring synMuv gene expression in a sample from said animal, an alteration in said expression relative to a sample from an unaffected animal being an indication that said animal has a cell proliferation disease or increased likelihood of developing a cell proliferation disease.

28. The method of claim 27, wherein said gene expression is measured by measuring the amount of synMuv polypeptide in said sample.

29. The method of claim 28, wherein said synMuv polypeptide is measured by immunological methods.

30. The method of claim 27, wherein said synMuv gene expression is measured by measuring the amount of synMuv RNA in said sample.

31. A method of identifying a gene which modulates cell proliferation, said method comprising:
(a) expressing in a cell (i) a first gene operably linked to a synMuv gene promoter; and (ii) a second candidate gene or a fragment thereof, and
(b) monitoring the expression of said first gene, wherein an increase in said expression identifies said candidate gene as a gene which modulates cell proliferation.

32. The method of claim 31, wherein said first gene is a synMuv gene.

33. A method of identifying a gene which modulates cell proliferation, said method comprising:
(a) expressing in a cell (i) at least a portion of a first gene selected from lin-9 and lin-15B; and (ii) a second candidate gene or a fragment thereof; and
(b) monitoring the expression of said first gene,
wherein an increase in said expression identifies said candidate gene as a gene which modulates cell proliferation.

34. A method of identifying a gene which modulates cell proliferation, said method comprising:
(a) expressing in a cell (i) at least a portion of a first gene, wherein said first gene comprises a sequence chosen from SEQ ID NOS: 17, 18, and 19; and (ii) a second candidate gene or a fragment thereof; and
(b) monitoring the expression of said first gene,
wherein an increase in said expression identifies said candidate gene as a gene which modulates cell proliferation.

35. A method of identifying a gene which modulates cell proliferation, said method comprising:
(a) expressing in a cell (i) at least a portion of a first gene operably linked to a promoter selected from the lin-9 promoter and the lin-15B promoter; and (ii) a second candidate gene or a fragment thereof; and
(b) monitoring the expression of said first gene,
wherein an increase in said expression identifies said candidate gene as a gene which modulates cell proliferation.

36. A method of diagnosing an animal for the presence of a cell proliferation disease or an increased likelihood of developing a cell proliferation disease, said method comprising measuring gene expression in a sample from said animal, wherein said gene hybridizes at high stringency to at least a portion of a sequence chosen from SEQ ID NOS: 17, 18, and 19, said portion being at least 50 nucleotides, and said polypeptide having at least one synMuv biological activity, an alteration in said expression relative to a sample from an unaffected animal being an indication that said animal has a cell proliferation disease or increased likelihood of developing a cell proliferation disease.

37. A method of diagnosing an animal for the presence of an cell proliferation disease or an increased likelihood of developing a cell proliferation disease, said method comprising
(a) isolating a sample of nucleic acid from said animal; and
(b) determining whether said nucleic acid comprises a mutated gene, wherein said gene comprises a sequence chosen from SEQ ID NOS: 17, 18, and 19,
a mutation in said nucleic acid being an indication that said animal has an cell proliferation disease or an increased likelihood of developing a cell proliferation disease.

38. A method of identifying a compound which modulates cell proliferation, said method comprising
(a) providing a cell expressing a gene operably linked to a gene promoter, said promoter selected from the list of the lin-9 promoter and the lin-15B promoter;
(b) contacting said cell with a candidate compound; and
(c) monitoring the expression of said gene, an alteration in the level of expression of said gene indicating the presence of a compound which modulates cell proliferation.
